(19) United States
(12) Patent Application Publication
(10) Pub. No.: US 2023/0399402 A1
(43) Pub. Date: Dec. 14, 2023

(54) HLA CLASS II-RESTRICTED TCRS AGAINST THE KRAS G12>V ACTIVATING MUTATION

(71) Applicants: La Jolla Institute for Immunology, La Jolla, CA (US); The Regents of the University of California, Oakland, CA (US)

(72) Inventors: Stephen P. Schoenberger, La Jolla, CA (US); Bjoern Peters, La Jolla, CA (US); Pandurangan Vijayanand, La Jolla, CA (US); Zeynep Kosalglu-Yalcin, La Jolla, CA (US); Aaron Miller, Oakland, CA (US); Ezra Cohen, Oakland, CA (US)

(21) Appl. No.: 18/035,160

(22) PCT Filed: Nov. 3, 2021
(86) PCT No.: PCT/US2021/057887

§ 371 Date: May 3, 2023

Related U.S. Application Data
(60) Provisional application No. 63/108,989, filed on Nov. 3, 2020.

(51) Int. Cl. C07K 16/28; C12N 5/0783; C12N 15/86; C07K 14/705; A61K 39/00

(52) U.S. Cl. CPC C07K 16/2809; C12N 5/0636; C12N 15/86; C07K 16/2863; C07K 14/70517; C07K 14/70578; A61K 39/4611; A61K 39/4632; A61K 39/4622; A61K 39/464411; A61K 39/4631; A61K 39/464404; C12N 2740/10043; C12N 2502/1107; C07K 2317/565; C07K 2317/24; C07K 2319/03; A61K 2239/13; A61K 2239/21

(57) ABSTRACT

The present invention includes engineered T cell receptor (TCR) proteins, nucleic acids, vectors, host cells, methods of treating cancer, and chimeric antigen receptor expressing T cell (CAR-T) comprising an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24, wherein the TCR is specific for a KRAS G12>V mutation peptide, antigen-MHC binding portions, and full length portions of the same.

### CROSS-REFERENCE TO RELATED APPLICATIONS

This application claims priority to U.S. Provisional Application Ser. No. 63/108,989, filed Nov. 3, 2020, the entire contents of which are incorporated herein by reference.

### TECHNICAL FIELD OF THE INVENTION

The present invention relates in general to the field of MHC-Class II restricted T cell Receptors, and more particularly, to T cell Receptors specific for the KRAS G12>V activating mutation.

### STATEMENT OF FEDERALLY FUNDED RESEARCH

Not applicable.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Nov. 3, 2021, is named LJII_2011WO_ST25.txt and is 106,963 bytes in size.

### BACKGROUND OF THE INVENTION

Without limiting the scope of the invention, its background is described in connection with cancers that have a KRAS mutation.

One such treatment is taught in U.S. Pat. No. 10,792,370, issued to Hu, entitled, “Antibody-drug conjugate”, which teaches an antibody-drug conjugate, in particular, to an antibody-drug conjugate targeting an epidermal growth factor receptor to target cancer cells that express a KRAS gene mutation or a BRAF gene mutation.

Another treatment is taught in U.S. Pat. No. 10,668,063, issued to Huang, entitled, “Method of treating cancer associated with a RAS mutation”, which teaches therapeutically treating a cancer characterized by expressing a mutant form of a RAS protein, comprising administering Plinabulin to a subject in need thereof, wherein the RAS protein is a mutant form of a NRAS protein, a HRAS protein, or a KRAS protein comprising one or more amino acid substitutions selected from the group consisting of G12C, G12R, G12D, G12V, G12F, G12L, G12N, G13C, G13R, G13S, G13A, G13V, G13P, S17G, P34S, A59E, A59G, A59T, Q61K, Q61L, Q61R, Q61H, K117N, A146P, A146T, A146V, and D153V.

Other patents teach treating KRAS cancers with using RNA interference. One such patent is U.S. Pat. No. 10,619,159, issued to Pecot, et al., entitled, “Methods and compositions using RNA interference for inhibition of KRAS,” which teaches inhibition of expression of mutant KRAS sequences using RNA interference by using double stranded RNA molecule comprising an antisense strand and a sense strand, wherein the nucleotide sequence of the antisense strand is at least 90% complementary to a region of the nucleotide sequence of a synthetic human KRAS gene that contains the missense mutations G12C, G12D, and G13D or the missense mutations G12C, G12V, and G13D, the region consisting essentially of about 18 to about 25 consecutive nucleotides; wherein the double stranded RNA molecule inhibits expression of a mutant human KRAS gene comprising one or more of the missense mutations G12C, G12D, G12V, and G13D and minimally inhibits expression of wild-type human KRAS.

Yet, despite these advances, a need remains for improved compositions and methods for treating KRAS gene mutation cancer cells that do not inhibit the activity of wild-type RAS.

### SUMMARY OF THE INVENTION

As embodied and broadly described herein, an aspect of the present disclosure relates to an engineered T cell receptor (TCR) comprising an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24, wherein the TCR is specific for a KRAS G12>V mutation peptide. In one aspect, the engineered TCR binds to the KRAS G12>V mutation peptide in a complex with HLA DRB5*01:01. In another aspect, the TCR comprises an alpha chain having at least 90, 95, 98, or 99% identity to the amino acid sequence of SEQ ID NO: 25, 27, 29, 31, 33, 35, 37, 39, 41, 43 and/or a beta chain having at least 90% identity to the amino acid sequence of SEQ ID NO: 26, 28, 30, 32, 34, 36, 38, 40, 42, and 44. In another aspect, the TCR is humanized. In another aspect, the TCR comprises an alpha chain having at least 90, 95, 98, or 99% identity to the nucleotide sequence of SEQ ID NO: 49, 51, 53, 55, 57, 59, 61, 63, 65, 67, 69, 71 and/or a beta chain having at least 95% identity to the nucleotide sequence of SEQ ID NO: 50, 52, 54, 56, 58, 60, 62, 64, 66, 68, 70, 72, and 74. In another aspect, the TCR is further defined as a soluble TCR, wherein the soluble TCR does not comprise a transmembrane domain, or comprises transmembrane domain that is a CD28 transmembrane domain or a CD8a transmembrane domain, or further comprises a T-cell signaling domain of any one of the following proteins: a human CD8-alpha protein, a human CD28 protein, a human CD3-zeta protein, a human FcRγ protein, a CD27 protein, an OX40 protein, a human 4-1BB protein, or any combination of the foregoing. In another aspect, the TCR further comprising a detectable label. In another aspect, the TCR is covalently bound to a therapeutic agent, an immunotoxin or a chemotherapeutic agent. In another aspect, the TCR does not recognize wild-type RAS, and the CDR3 is selected from SEQ ID NO: 1, 3, 5 and a beta chain CDR3 having the amino acid sequence of SEQ ID NO: 2, 4, 6. In another aspect, the TCR is part of a multivalent TCR complex comprising a plurality of TCRs according to claim 1. In another aspect, the multivalent TCR comprises 2, 3, 4 or more TCRs associated with one another; wherein the multivalent TCR is present in a lipid bilayer, in a liposome, or is attached to a nanoparticle; or wherein the TCRs are associated with one another via a linker molecule.

In another embodiment, a polypeptide encoding the TCR comprising an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24 is disclosed, wherein the TCR is specific for a KRAS G12>V mutation peptide.

In another embodiment, a polynucleotide encoding TCR polypeptide(s) comprising an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24 is disclosed, wherein the TCR is specific for a KRAS G12>V mutation peptide.

In another embodiment, an expression vector encoding TCR polypeptide(s) comprising an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24 is disclosed, wherein the TCR is specific for a KRAS G12>V mutation peptide. In one aspect, the sequence encoding the TCR is under the control of a promoter. In another aspect, the expression vector is a viral or a retroviral vector. In another aspect, the vector further encodes a linker domain positioned between the alpha chain and beta chain. In another aspect, the linker domain comprises one or more protease cleavage sites, or wherein the one or more cleavage sites are separated by a spacer.

In another embodiment, a host cell engineered to express a polypeptide encoding the TCR comprising an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24 is disclosed, wherein the TCR is specific for a KRAS G12>V mutation peptide. In another aspect, the cell is a T cell, NK cell, invariant NK cell, NKT cell, mesenchymal stem cell (MSC), or induced pluripotent stem (iPS) cell. In another aspect, the host cell is an immune cell. In another aspect, the T cell is a CD8⁺ T cell, CD4⁺ T cell, or γδ T cell. In another aspect, the T cell is a regulatory T cell (Treg). In another aspect, the host cell is autologous or allogeneic.

In another embodiment, a method is provided for engineering a host cell comprising contacting an immune cell with the TCR or the expression vector of the present disclosure. In one aspect, the method comprises contacting is further defined as transfecting or transducing, wherein transfecting comprises electroporating RNA encoding the TCR described hereinabove into the immune cell.

In another embodiment, a method for treating a subject with a cancer is disclosed, comprising a KRAS G12>V mutation peptide, the method comprising: administering to the subject an effective amount of one or more immune cells modified by cloning genes of the alpha and beta chains of a T cell receptor (TCR) ex vivo to express a chimeric antigen receptor specific for the KRAS G12>V mutation, wherein the chimeric antigen receptor comprises an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24. In one aspect, the immune cell is T cell, NK cell, invariant NK cell, NKT cell, mesenchymal stem cell (MSC), or induced pluripotent stem (iPS) cell, or a peripheral blood lymphocyte. In another aspect, method further comprises at least one of: sorting the immune cells into T cells to isolate TCR engineered T cells; performing a T cell cloning of the immune cells by serial dilution; or expanding a T cell clone from the immune cells by a rapid expansion protocol. In another aspect, the subject is identified to have an HLA DRB5*01:01 allele. In another aspect, the immune cell is a T cell selected from a CD8⁺ T cell, CD4⁺ T cell, or Treg. In another aspect, the cancer is selected from colorectal cancer, pancreatic cancer, renal cancer, lung cancer, liver cancer, breast cancer, prostate cancer, gastrointestinal cancer, peritoneal cancer, melanoma, endometrial cancer, ovarian cancer, cervical cancer, uterine carcinoma, bladder cancer, glioblastoma, brain metastases, salivary gland carcinoma, thyroid cancer, brain cancer, lymphoma, myeloma, and head and neck cancer. In another aspect, the cancer is selected from pancreatic ductal adenocarcinoma and colorectal adenocarcinoma. In another aspect, the TCR engineered cells are autologous or allogeneic. In another aspect, the method further comprises administering a second anticancer selected from chemotherapy, immunotherapy, surgery, radiotherapy, or biotherapy. In another aspect, the one or more immune cells are administered intravenously, intraperitoneally, intratracheally, intratumorally, intramuscularly, endoscopically, intralesionally, percutaneously, subcutaneously, regionally, or by direct injection or perfusion.

In another embodiment, a chimeric antigen receptor expressing T cell (CAR-T) is disclosed comprising an antigen recognition moiety and a T-cell activation moiety, wherein the T-cell activation moiety comprises a transmembrane domain, and wherein the antigen recognition moiety is directed against a KRAS G12>V mutation. In another aspect, the antigen recognition moiety does not recognize non-mutated RAS. In another aspect, the transmembrane domain is a CD28 transmembrane domain or a CD8a transmembrane domain. In another aspect, the T-cell activation moiety comprises a T-cell signaling domain of any one of the following proteins: a human CD8-alpha protein, a human CD28 protein, a human CD3-zeta protein, a human FcRγ protein, a CD27 protein, an OX40 protein, a human 4-1BB protein, or any combination of the foregoing. In another aspect, the antigen recognition moiety comprises the amino acid sequence of wherein the TCR comprises an alpha chain variable region having at least 90, 95, 98, or 99% identity to the amino acid sequence of SEQ ID NO: 25, 27, 29, 31, 33, 35, 37, 39, 41, 43 and/or a beta chain variable region having at least 90% identity to the amino acid sequence of SEQ ID NO: 26, 28, 30, 32, 34, 36, 38, 40, 42, and 44. In another aspect, the antigen recognition moiety comprises an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24.

### BRIEF DESCRIPTION OF THE DRAWINGS

For a more complete understanding of the features and advantages of the present invention, reference is now made to the detailed description of the invention along with the accompanying figures and in which:

FIGS. 1A to 1D shows the results from three novel KRAS^{G12V}-reactive TCRs recognize distinct mutant epitopes, but only TCR2 recognizes processed and presented neoantigen.

FIGS. 2A to 2D shows the coreceptor dependence, avidity, and restriction characteristics for TCR2.

FIGS. 3A and 3B show that TCR2 recognizes and kills tumor cells directly. An inducible cancer stem cell (iCSC) from PT37 which matches PT66 restriction requirements for TCR2, but lacks the KRAS^{G12V} mutation.

FIG. 4 shows the MHC restriction of the T cell receptors of the present disclosure.

### DETAILED DESCRIPTION OF THE INVENTION

A detailed description of one or more embodiments of the invention is provided below along with accompanying figures that illustrate the principles of the invention. The invention is described in connection with such embodiments, but the invention is not limited to any embodiment. The scope of the invention is limited only by the claims. While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “chimeric antigen receptors (CARs),” as used herein, may refer to artificial T cell receptors, chimeric T cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen, for example. In some embodiments, CARs comprise an intracellular activation domain, a transmembrane domain, and an extracellular domain comprising a tumor associated antigen binding region. In particular aspects, CARs comprise fusions of single-chain variable fragments (scFv) derived from monoclonal antibodies, fused to CD3-zeta a transmembrane domain and endodomain. The specificity of other CAR designs may be derived from ligands of receptors (e.g., peptides) or from pattern-recognition receptors, such as Dectins. In certain cases, the spacing of the antigen-recognition domain can be modified to reduce activation-induced cell death. In certain cases, CARs comprise domains for additional co-stimulatory signaling, such as CD3ζ, FcR, CD27, CD28, CD137, DAP10, and/or OX40. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

As used herein, the term “chimeric antigen receptor T cell” or “CAR-T” refers to a T cell that has been modified to express the TCR of the present disclosure. Non-limiting examples of T cells that can be made into CAR-T cells include: autologous or allogeneic T cells, or even, regulatory T cells, CD4+ T cells, CD8+ T cells, gamma-delta T cells, NK cells, invariant NK cells, NKT cells, mesenchymal stem cell, or pluripotent stem cells.

As used herein, the term “essentially free,” refers to a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein, the terms “inhibiting,” “reducing,” or “prevention,” or any variation of these terms, when used in the claims and/or the specification includes any measurable decrease or complete inhibition to achieve a desired result.

As used herein, the term “effective” refers to an amount of the present disclosure that is adequate to accomplish a desired, expected, or intended result.

As used herein, the terms “immune system cell” or “immune cell” refer to any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells and natural killer (NK) cells, including Natural Killer T (NK-T) cells). Exemplary immune system cells include a CD4⁺ T cell, a CD8⁺ T cell, a CD4⁻CD8⁻ double negative T cell, a γδ T cell, a regulatory T cell, a natural killer cell, a natural killer T cell, and a dendritic cell. Macrophages and dendritic cells may be referred to as “antigen presenting cells” or “APCs,” which are specialized cells that can activate T cells when a major histocompatibility complex (MEW) receptor on the surface of the APC complexed with a peptide interacts with a TCR on the surface of a T cell.

As used herein, the term “Major Histocompatibility Complex” (MHC) refers to glycoproteins that deliver peptide antigens to a cell surface. MHC class I molecules are heterodimers having a membrane spanning a chain (with three a domains) and a non-covalently associated β2 microglobulin. MHC class II molecules are composed of two transmembrane glycoproteins, α and β, both of which span the membrane. Each chain has two domains. MHC class I molecules deliver peptides originating in the cytosol to the cell surface, where a peptide:MHC complex is recognized by CD8⁺ T cells. MHC class II molecules deliver peptides originating in the vesicular system to the cell surface, where a peptide:MHC complex is recognized by CD4⁺ T cells. Human MHC is referred to as human leukocyte antigen (HLA). HLA-II types include DP, DM, DOA, DOB, DQ, and DR. Numerous alleles encoding the subunits of the various HLA types are known, including, for example, HLA-DQA1*03, HLA-DQB1*0301, HLA-DQB1*0302, HLA-DQB1*0303.

As used herein, the terms “T cell” or “T lymphocyte” refers to an immune system cell that matures in the thymus and produces T cell receptors (TCRs). T cells can be naive (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of: central memory T cells (T_{CM}, increased expression of CD62L, CCR7, CD28, CD127, CD45RO, and CD95, and decreased expression of CD54RA as compared to naive T cells); and effector memory T cells (T_{EM}, decreased expression of CD62L, CCR7, CD28, CD45RA, and increased expression of CD127 as compared to naive T cells or T_{CM}).

As used herein, “effector T cells” (T_{E}) refers to antigen-experienced CD8⁺ cytotoxic T lymphocytes that have decreased expression of CD62L, CCR7, CD28, and are positive for granzyme and perform as compared to T_{CM}. Helper T cells (T_{H}) are CD4+ cells that influence the activity of other immune cells by releasing cytokines. CD4⁻ T cells can activate and suppress an adaptive immune response, and which of those two functions is induced will depend on presence of other cells and signals. T cells can be collected using known techniques, and the various subpopulations or combinations thereof can be enriched or depleted by known techniques, such as by affinity binding to antibodies, flow cytometry, or immunomagnetic selection. Other exemplary T cells include regulatory T cells, such as CD4⁺CD25⁺ (Foxp3⁺) regulatory T cells and Treg17 cells, as well as Tr1, Th3, CD8⁺CD28⁻, and Qa-1 restricted T cells.

As used herein, the term “T cell receptor” (TCR) refers to an immunoglobulin superfamily member having a variable binding domain, a constant domain, a transmembrane region, and a short cytoplasmic tail (see, e.g., Janeway et al., Immunobiology: The Immune System in Health and Disease, 3.sup.rd Ed., Current Biology Publications, p. 4:33, 1997, relevant portions incorporated herein by reference) capable of specifically binding to an antigen peptide bound to, or presented by, a MHC. A TCR can be found on the surface of a cell or in soluble form and generally is comprised of a heterodimer having α and β chains (also known as TCRα and TCRβ, respectively), or γ and δ chains (also known as TCRγ and TCRδ, respectively). Like immunoglobulins, the extracellular portion of TCR chains (e.g., α-chain, β-chain) contain two immunoglobulin domains: a variable domain (e.g., α-chain variable domain or Vα, β-chain variable domain or Vβ; typically amino acids 1 to 116 based on Kabat numbering (Kabat et al., “Sequences of Proteins of Immunological Interest, US Dept. Health and Human Services, Public Health Service National Institutes of Health, 1991, 5^{th} ed., relevant portions incorporated herein by reference) at the N-terminus; and one constant domain (e.g., α-chain constant domain or Cα, typically amino acids 117 to 259 based on Kabat, β-chain constant domain or Cβ, typically amino acids 117 to 295 based on Kabat) adjacent to the cell membrane. Also, like immunoglobulins, the variable domains contain complementary determining regions (CDRs) separated by framework regions (FRs) (see, e.g., Jones et al., Proc. Nat'l Acad. Sci. U.S.A. 87:9138, 1990; Chothia et al., EMBO 1 7:3745, 1988; see also Lefranc et al., Dev. Comp. Immunol. 27:55, 2003, relevant portions incorporated herein by reference). TCR variable domain sequences can be aligned to a numbering scheme (e.g., Kabat, EU, International Immunogenetics Information System (IMGT) and Aho), which can allow equivalent residue positions to be annotated and for different molecules to be compared using Antigen receptor Numbering And Receptor Classification (ANARCI) software tool (2016, Bioinformatics 15:298-300, relevant portions incorporated herein by reference). A numbering scheme provides a standardized delineation of framework regions and CDRs in the TCR variable domains.

In certain embodiments, a TCR is found on the surface of T cells (or T lymphocytes) and associates with the CD3 complex. The source of a TCR as used in the present disclosure may be from various animal species, such as a human, mouse, rat, rabbit or other mammal.

In certain embodiments, the present disclosure provides an engineered T cell receptor (TCR) comprising an alpha chain having at least 90, 95, 98, or 99% identity to the amino acid sequence of SEQ ID NO: 25, 27, 29, 31, 33, 35, 37, 39, 41, 43 and/or a beta chain having at least 90% identity to the amino acid sequence of SEQ ID NO: 26, 28, 30, 32, 34, 36, 38, 40, 42, and 44. In particular aspects, the TCR has an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24.

In certain embodiments, the TCR comprises the TCR comprises an alpha chain having at least 90, 95, 98, or 99% identity to the nucleotide sequence of SEQ ID NO: 49, 51, 53, 55, 57, 59, 61, 63, 65, 67, 69, 71 and/or a beta chain having at least 95% identity to the nucleotide sequence of SEQ ID NO: 50, 52, 54, 56, 58, 60, 62, 64, 66, 68, 70, 72, and 74.

As used herein, the term “CD3” refers to a multi-protein complex of six chains (see, Abbas and Lichtman, 2003; Janeway et al., p. 172 and 178, 1999, relevant portions incorporated herein by reference). In mammals, the complex comprises a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif (ITAM), whereas each CD3ζ chain has three ITAMs. ITAMs are important for the signaling capacity of a TCR complex. CD3 as used in the present disclosure may be from various animal species, including human, mouse, rat, or other mammals.

As used herein, the term “TCR complex” refers to a complex formed by the association of CD3 with TCR. For example, a TCR complex can be composed of a CD3γ chain, a CD3δ chain, two CD3ε chains, and a homodimer of CD3ζ chains, a TCRα chain, and a TCRβ chain. Alternatively, a TCR complex can be composed of a CD3γ chain, a CD3δ chain, two CD3ε chains, and a homodimer of CD3ζ chains, a TCRγ chain, and a TCRδ chain.

As used herein, the term “component of a TCR complex,” refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRα, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

As used herein, the term “CD4” refers to an immunoglobulin co-receptor glycoprotein that assists the TCR in communicating with antigen-presenting cells (see, Campbell & Reece, Biology 909 (Benjamin Cummings, Sixth Ed., 2002); UniProtKB P01730, relevant portions incorporated herein by reference). CD4 is found on the surface of immune cells such as T helper cells, monocytes, macrophages, and dendritic cells, and includes four immunoglobulin domains (D1 to D4) that are expressed at the cell surface. During antigen presentation, CD4 is recruited, along with the TCR complex, to bind to different regions of the MHCII molecule (CD4 binds MHCII β2, while the TCR complex binds MHC-II α1/β1). Without wishing to be bound by theory, it is believed that close proximity to the TCR complex allows CD4-associated kinase molecules to phosphorylate the immunoreceptor tyrosine activation motifs (ITAMs) present on the cytoplasmic domains of CD3. This activity is thought to amplify the signal generated by the activated TCR in order to produce various types of T helper cells.

As used herein, the term “CD8 co-receptor” or “CD8” means the cell surface glycoprotein CD8, either as an alpha-alpha homodimer or an alpha-beta heterodimer. The CD8 co-receptor assists in the function of cytotoxic T cells (CD8⁺) and functions through signaling via its cytoplasmic tyrosine phosphorylation pathway (Gao and Jakobsen, Immunol. Today 21:630-636, 2000; Cole and Gao, Cell. Mol. Immunol. 1:81-88, 2004). In humans, there are five (5) different CD8 beta chains (see UniProtKB identifier P10966) and a single CD8 alpha chain (see UniProtKB identifier P01732, relevant portions incorporated herein by reference).

As used herein, the terms “variable region” or “variable domain” refers to the domain of a TCR α-chain or β-chain (or γ-chain and δ-chain for γδ TCRs) that is involved in binding of the TCR to antigen. The variable domains of the α-chain and β-chain (Vα and Vβ, respectively) of a native TCR generally have similar structures, with each domain comprising four generally conserved framework regions (FRs) and three CDRs. The Vα domain is encoded by two separate DNA segments, the variable gene segment and the joining gene segment (V-J); the Vβ domain is encoded by three separate DNA segments, the variable gene segment, the diversity gene segment, and the joining gene segment (V-D-J). In certain cases, a single Vα or Vβ domain may be sufficient to confer antigen-binding specificity. TCRs that bind a particular antigen may be isolated using a Vα or Vβ domain from a TCR that binds the antigen to screen a library of complementary Vα or Vβ domains, respectively.

As used herein, the terms “complementarity determining region,” and “CDR,” are synonymous with “hypervariable region”, and refer to non-contiguous sequences of amino acids within TCR variable regions that confer antigen specificity and/or binding affinity to the TCR. In general, there are three CDRs in each α-chain variable region αCDR1, αCDR2, αCDR3) and three CDRs in each β-chain variable region (βCDR1, βCDR2, βCDR3). CDR3 is thought to be the main CDR responsible for recognizing processed antigen. CDR1 and CDR2 mainly interact with the MHC. In certain embodiments, a binding protein of the present disclosure comprises an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24. The CRD1 and CDR2 of the alpha and beta chains can be found in the full-length sequences, respectively, wherein an alpha chain having at least 90, 95, 98, or 99% identity to the amino acid sequence of SEQ ID NO: 25, 27, 29, 31, 33, 35, 37, 39, 41, 43 and/or a beta chain having at least 90% identity to the amino acid sequence of SEQ ID NO: 26, 28, 30, 32, 34, 36, 38, 40, 42, and 44.

As used herein, the terms “Antigen” or “Ag” refer to an immunogenic molecule that provokes an immune response. This immune response may involve antibody production, activation of specific immunologically-competent cells (e.g., T cells), or both. An antigen (immunogenic molecule) may be, for example, a peptide, glycopeptide, polypeptide, glycoprotein, polynucleotide, polysaccharide, lipid or the like. An antigen can be synthesized, produced recombinantly, or derived from a biological sample. Biological samples that contain antigens can include tissue samples, tumor samples, cells, biological fluids, or combinations thereof. Antigens can be produced by cells that have been modified or genetically engineered to express an antigen. The antigen herein is KRAS G12>V mutation peptide.

As used herein, the terms “epitope” or “antigenic epitope” refer to any molecule, structure, amino acid sequence or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three-dimensional structural characteristics, as well as specific charge characteristics.

As used herein, the term “binding domain” (also referred to as a “binding region” or “binding moiety”), refers to a molecule or portion thereof (e.g., peptide, oligopeptide, polypeptide, protein) that possesses the ability to specifically and non-covalently associate, unite, or combine with a target (e.g., KRAS G12>V mutation peptide). A binding domain includes any naturally occurring, synthetic, semi-synthetic, or recombinantly produced binding partner for a biological molecule, a molecular complex (i.e., complex comprising two or more biological molecules), or other target of interest. Exemplary binding domains include single chain immunoglobulin variable regions (e.g., scTCR, scFv), receptor ectodomains, ligands (e.g., cytokines, chemokines), or synthetic polypeptides selected for their specific ability to bind to a biological molecule, a molecular complex or other target of interest.

In certain embodiments, a receptor or binding domain may have “enhanced affinity,” which refers to selected or engineered receptors or binding domains with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof. In certain embodiments, enhanced affinity TCRs may be codon optimized to enhance expression in a particular host cell, such as T cells (Scholten et al., Clin. Immunol. 119:135, 2006, relevant portions incorporated herein by reference).

A variety of assays are known for identifying binding domains of the present disclosure that specifically bind a particular target, as well as determining binding domain or fusion protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, relevant portions incorporated herein by reference). Assays for assessing affinity or apparent affinity or relative affinity are also known.

As used herein, the term “KRAS G12>V mutation peptide” or “KRAS G12>V antigen peptide” refers to a protein or polypeptide that is a KRAS G12>V peptide:HLA complex, e.g., on a cell surface.

As used herein, the term “antigen processing” refers to the processing of a protein into peptides for presentation by antigen presenting cells (APC) (such as dendritic cells, macrophages, lymphocytes or other cell types), and of antigen presentation by APC to T cells, including major histocompatibility complex (MHC)-restricted presentation between immunocompatible (e.g., sharing at least one allelic form of an MHC gene that is relevant for antigen presentation) APC and T cells, are well established (see, e.g., Murphy, Janeway's Immunobiology (8^{th} Ed.) 2011 Garland Science, NY; chapters 6, 9 and 16, relevant portions incorporated herein by reference). For example, processed antigen peptides originating in the cytosol (e.g., tumor antigen, intracellular pathogen) are generally from about 7 amino acids to about 11 amino acids in length and will associate with class I MEW molecules, whereas peptides processed in the vesicular system (e.g., bacterial, viral) will generally vary in length from about 10 amino acids to about 25 amino acids and associate with class II MHC molecules.

As used herein, the terms “nucleic acid” or “nucleic acid molecule” refer to any of deoxyribonucleic acid (DNA), ribonucleic acid (RNA), oligonucleotides, fragments generated, for example, by the polymerase chain reaction (PCR) or by in vitro translation, and fragments generated by any of ligation, scission, endonuclease action, or exonuclease action. In certain embodiments, the nucleic acids of the present disclosure are produced by PCR. Nucleic acids may be composed of monomers that are naturally occurring nucleotides (such as deoxyribonucleotides and ribonucleotides), analogs of naturally occurring nucleotides (e.g., α-enantiomeric forms of naturally occurring nucleotides), or a combination of both. Modified nucleotides can have modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acid molecules can be either single stranded or double stranded.

As used herein, the term “isolated” refers to a material that is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide.

As used herein, the term “gene” refers to a segment of DNA involved in producing a polypeptide chain and can includes regions preceding and following the coding region as well as intervening sequences (introns) between individual coding segments (exons).

As used herein, the term “recombinant” refers to a cell, microorganism, nucleic acid molecule, or vector that has been genetically engineered by human intervention-13 that is, modified by introduction of a heterologous nucleic acid molecule, or refers to a cell or microorganism that has been altered such that expression of an endogenous nucleic acid molecule or gene is controlled, deregulated, deleted, attenuated, or constitutive. Human generated genetic alterations may include, for example, modifications that introduce nucleic acid molecules (which may include an expression control element, such as a promoter) that encode one or more proteins or enzymes, or other nucleic acid molecule additions, deletions, substitutions, or other functional disruption of or addition to a cell's genetic material. Exemplary modifications include those in coding regions or functional fragments thereof of heterologous or homologous polypeptides from a reference or parent molecule.

As used herein, the term “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s). In certain embodiments, a mutation is a substitution of one or three codons or amino acids, a deletion of one to about 5 codons or amino acids, or a combination thereof.

As used herein, the term a “conservative substitution” refers to a substitution of one amino acid for another amino acid that has similar properties. Exemplary conservative substitutions are well known in the art (see, e.g., WO 97/09433 at page 10; Lehninger, Biochemistry, 2.sup.nd Edition; Worth Publishers, Inc. NY, N.Y., pp. 71-77, 1975; Lewin, Genes IV, Oxford University Press, NY and Cell Press, Cambridge, Ma., p. 8, 1990, relevant portions incorporated herein by reference).

As used herein, the term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, an RNA vector or a linear or circular DNA or RNA molecule that may include chromosomal, non-chromosomal, semi-synthetic or synthetic nucleic acid molecules. Generally, vectors are those capable of autonomous replication (episomal vector) or expression of nucleic acid molecules to which they are linked (expression vectors). Viral vectors can include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996, relevant portions incorporated herein by reference).

As used herein, the term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably-linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

As used herein, the term “expression” refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof.

As used herein, the term “introduced” in the context of inserting a nucleic acid molecule into a cell, is also known to be achieved by “transfection”, or “transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

As used herein, a “heterologous” nucleic acid molecule, construct or sequence refers to a nucleic acid molecule or portion of a nucleic acid molecule that is not native to a host cell but may be homologous to a nucleic acid molecule or portion of a nucleic acid molecule from the host cell. The source of the heterologous nucleic acid molecule, construct or sequence may be from a different genus or species. In certain embodiments, a heterologous nucleic acid molecule is added (i.e., is not endogenous or native) to a host cell or host genome by, for example, conjugation, transformation, transfection, electroporation, or the like, wherein the added molecule may integrate into the host genome or exist as extra-chromosomal genetic material (e.g., as a plasmid or other form of self-replicating vector), and may be present in multiple copies. In addition, “heterologous” refers to a non-native enzyme, protein or other activity encoded by a heterologous polynucleotide introduced into the host cell, even if the host cell encodes a homologous protein or activity.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. For example, as disclosed herein, a host cell can be modified to express two or more heterologous nucleic acid molecules encoding desired binding proteins specific for a KRAS G12>V antigen peptide (e.g., TCRα and TCRβ). When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules, or protein activities, refer to the number of encoding nucleic acid molecules or the number of protein activities, not the number of separate nucleic acid molecules introduced into a host cell.

As used herein, the term “endogenous” or “native” refers to a gene, protein, or activity that is normally present in a host cell. Moreover, a gene, protein or activity that is mutated, overexpressed, shuffled, duplicated or otherwise altered as compared to a parent gene, protein or activity is still considered to be endogenous or native to that particular host cell. For example, an endogenous control sequence from a first gene (e.g., promoter, translational attenuation sequences) may be used to alter or regulate expression of a second native gene or nucleic acid molecule, wherein the expression or regulation of the second native gene or nucleic acid molecule differs from normal expression or regulation in a parent cell.

As used herein, the terms “homologous” or “homolog” refer to a molecule or activity found in or derived from a host cell, species or strain. For example, a heterologous polynucleotide may be homologous to a native host cell gene, and may optionally have an altered expression level, a different sequence, an altered activity, or any combination thereof.

As used herein, the term “sequence identity,” refers to the percentage of amino acid residues (or a polynucleotide) in one sequence that are identical with the amino acid residues (or a polynucleotide) in another reference polypeptide sequence (or a polynucleotide sequence) after aligning the sequences and introducing gaps, if necessary, to achieve the maximum percent sequence identity, and not considering any conservative substitutions as part of the sequence identity. The percentage sequence identity values can be generated using the NCBI BLAST2.0 software as defined by Altschul et al. (1997) “Gapped BLAST and PSI-BLAST: a new generation of protein database search programs”, Nucleic Acids Res. 25:3389-3402, with the parameters set to default values, relevant portions incorporated herein by reference.

As used herein, a “hematopoietic progenitor cell” is a cell that can be derived from hematopoietic stem cells (such as bone marrow or fetal tissue) that is capable of further differentiation into mature cells types (e.g., immune system cells). Exemplary hematopoietic progenitor cells include those with a CD24^{lo}Lin⁻CD117⁺ phenotype or those found in the thymus (referred to as progenitor thymocytes).

As used herein, the term “host” refers to a cell (e.g., a T cell) such as a mammalian insect, plant, yeast, or microorganism targeted for genetic modification with a heterologous nucleic acid molecule to produce the T cell Receptor alpha and/or beta chain polypeptide(s) of interest, specifically, an KRAS G12>V mutation antigen peptide specific TCR when seen in the context of Class II MHC. In certain embodiments, a host cell may optionally possess or be modified to include other genetic modifications that confer desired properties related or unrelated to biosynthesis of the T cell Receptor alpha and/or beta chain polypeptide(s) of interest, specifically, an KRAS G12>V mutation antigen peptide specific TCR. In certain embodiments, a host cell is a human hematopoietic progenitor cell transduced with a heterologous nucleic acid molecule encoding a TCRα/TCRβ chain specific for a KRAS G12>V mutation antigen peptide.

T Cell Receptor (TCR). The genetically engineered antigen receptors include recombinant T cell receptors (TCRs) and/or TCRs cloned from naturally occurring T cells.

As used herein, the term “T cell receptor” or “TCR” refers to a molecule that includes a variable α and β chains (also known as TCRα and TCRβ, respectively) or a variable γ and δ chains (also known as TCRγ and TCRδ, respectively) and that is capable of specifically binding to a KRAS G12>V mutation antigen peptide bound to a MHC receptor. In some embodiments, the TCR is in the αβ form. In certain embodiments, the engineered TCR has an alpha chain CDR3 of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24. In some embodiments, the TCR comprises an alpha chain having at least 90, 95, 98, or 99% identity to the nucleotide sequence of SEQ ID NO: 49, 51, 53, 55, 57, 59, 61, 63, 65, 67, 69, 71 and/or a beta chain having at least 95% identity to the nucleotide sequence of SEQ ID NO: 50, 52, 54, 56, 58, 60, 62, 64, 66, 68, 70, 72, and 74, respectively.

Generally, TCRs exist in αβ and γδ forms, but T cells expressing them may have distinct locations in the body and/or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, Immunobiology: The Immune System in Health and Disease, 3.sup.rd Ed., Current Biology Publications, p. 433, 1997), relevant portions incorporated herein by reference. For example, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. As used herein, the term “TCR” should be understood to also include functional TCR fragments thereof. The term also encompasses intact or full-length TCRs, including TCRs in the αβ form or γδ form when having the CDR1,2, and/or 3 disclosed herein.

As used herein, an “antigen-binding portion” or “antigen-binding fragment” of a TCR, which are used interchangeably, refers to a molecule that contains a portion of the structural domains of a TCR and that binds the antigen in the context of an MHC protein, referred to as an MHC-peptide complex, to which the full TCR binds. An antigen-binding portion contains the variable domains of a TCR, such as variable α chain and variable β chain of a TCR, sufficient to form a binding site for binding to a specific MHC-peptide complex, such as generally where each chain contains three complementarity determining regions.

It is known that the variable domains of the TCR chains associate to form complementarity determining regions (CDRs) analogous to immunoglobulins, which confer antigen recognition and determine peptide specificity by forming the binding site of the TCR molecule, determine peptide specificity, and determine the MHC molecules that forms the peptide-MHC complex. Like immunoglobulins, TCR CDRs are separated by framework regions (FRs) (see, e.g., Jores et al., PNAS U.S.A. 87:9138, 1990; Chothia et al., EMBO J. 7:3745, 1988; see also Lefranc et al., Dev. Comp. Immunol. 27:55, 2003), relevant portions incorporated herein by reference. In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or Cₐ, typically amino acids 117 to 259 based on Kabat, β-chain constant domain or Cp, typically amino acids 117 to 295 based on Kabat) adjacent to the cell membrane, relevant portions incorporated herein by reference. Generally, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond linking the two chains. In certain examples, the TCR may have an additional cysteine residue in each of the α and β chains such that the TCR contains two disulfide bonds in the constant domains.

Generally, TCR chains contain a transmembrane domain, although that can be removed, or replaced with other transmembrane domain(s). Often, the transmembrane domain is positively charged. Generally, TCR contains a cytoplasmic tail, although that can be removed, or replaced with other cytoplasmic tail(s). Generally, the structure allows the TCR to associate with other molecules of the CD3 complex. For example, the TCR containing constant domains with a transmembrane region can anchor the protein in the cell membrane and associate with invariant subunits of the CD3 signaling complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell.

Generally, the TCR may be a heterodimer of two chains α and β (or γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T-cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15: 169-180 and Cohen et al. (2005) J Immunol. 175:5799-5808). In some embodiments, phage display is used to isolate TCRs against a target antigen (see, e.g., Varela-Rohena et al. (2008) Nat Med. 14: 1390-1395 and Li (2005) Nat Biotechnol. 23:349-354, relevant portions incorporated herein by reference). In some example, the TCR or antigen-binding portion thereof is synthetically generated from knowledge of the sequence of the TCR.

Chimeric T Cell Receptors. The present disclosure also includes engineered antigen receptors include chimeric antigen receptors (CARs), including activating or stimulatory CARs, costimulatory CARs (see WO2014/055668), and/or inhibitory CARs (iCARs, see Fedorov et al., Sci. Transl. Medicine, 5(215) (December, 2013), relevant portions incorporated herein by reference. The CARs generally include an extracellular antigen (or ligand) binding domain linked to one or more intracellular signaling components, and in some aspects, via linkers and/or transmembrane domain(s). Generally, these molecules mimic or approximate a signal through a natural antigen receptor, a signal through such a receptor in combination with a costimulatory receptor, and/or a signal through a costimulatory receptor alone. The T cell CAR can be used to replace an antigen-binding portion or portions of an antibody molecule, to make a single-chain antibody fragment (scFv) derived from the variable heavy (VH) and variable light (VL) chains of a monoclonal antibody (mAb).

The arrangement of the antigen-binding domain of a CAR may be multimeric, such as dimeric or multimeric. The multimers can be formed by cross pairing of the variable portions of the light and heavy chains. A hinge portion of the CAR may be shortened or deleted to make a CAR with a single antigen binding domain, a transmembrane region and an intracellular signaling domain. Also, the Fc portion of an antibody may be deleted from scFv used to as an antigen-binding region to generate CARs according to the present disclosure. In some embodiments, an antigen-binding region may encode just one of the Fc domains, e.g., either the CH2 or CH3 domain from human immunoglobulin. One may also include the hinge, CH2, and CH3 region of a human immunoglobulin that has been modified to improve dimerization and oligomerization. In some embodiments, the hinge portion of may comprise or consist of a 8-14 amino acid peptide (e.g., a 12 AA peptide), a portion of CD8α, or the IgG4 Fc. In some embodiments, the antigen binding domain may be suspended from cell surface using a domain that promotes oligomerization, such as CD8α.

The intracellular signaling domain of a CAR will generally cause the activation of at least one of the normal effector functions of an immune cell comprising the CAR. For example, the intracellular domain may promote an effector function of a T cell such as, e.g., cytolytic activity or helper activity including the secretion of cytokines. The effector function in a naive, memory, or memory-type T cell may include antigen-dependent proliferation. As used herein, the term “intracellular signaling domain” refers to the portion of a CAR that can transduce the effector function signal and/or direct the cell to perform a specialized function. While usually the entire intracellular signaling domain may be included in a CAR, in some cases a truncated portion of a cytoplasmic domain may be included.

For example, the TCR intracellular domains may be engineered to have the zeta chain of the T cell receptor or any of its homologs (e.g., zeta, delta, gamma, or epsilon), MB1 chain, B29, Fc RIII, Fc RI, and combinations of signaling molecules, such as CD3C and CD28, CD27, 4-1BB, DAP-10, OX40, and combinations thereof, as well as other similar molecules and fragments. Intracellular signaling portions of other members of the families of activating proteins can be used, such as FcγRIII and FcεRI. Examples of these alternative transmembrane and intracellular domains can be found, e.g., Gross et al. (1992), Stancovski et al. (1993), Moritz et al. (1994), Hwu et al. (1995), Weijtens et al. (1996), and Hekele et al. (1996), which are incorporated herein by reference in their entirety. The transmembrane and/or intracellular domain may include a sequence encoding a costimulatory receptor such as, e.g., a modified CD28 intracellular signaling domain, CD28, CD27, OX-40 (CD134), DAP10, or 4-1BB (CD137) costimulatory receptor. In some embodiments, both a primary signal initiated by CD3ζ, an additional signal provided by a human costimulatory receptor may be included in a CAR to more effectively activate transformed T cells, which may help improve in vivo persistence and the therapeutic success of the adoptive immunotherapy. The CAR may be engineered with transmembrane domain(s), e.g., the human IgG4 Fc hinge and Fc regions, the human CD4 transmembrane domain, the human CD28 transmembrane domain, the transmembrane human CD3ζ domain, or a cysteine mutated human CD3ζ domain, or a transmembrane domains from a human transmembrane signaling protein such as, e.g., the CD16 and CD8 and erythropoietin receptor.

An isolated nucleic acid segment and expression cassette including DNA sequences that encode a CAR may be generated that include a TCR alpha chain having at least 90, 95, 98, or 99% identity to the nucleotide sequence of SEQ ID NO: 49, 51, 53, 55, 57, 59, 61, 63, 65, 67, 69, 71 and/or a beta chain having at least 95% identity to the nucleotide sequence of SEQ ID NO: 50, 52, 54, 56, 58, 60, 62, 64, 66, 68, 70, 72, and 74. A variety of vectors may be used for delivery of the DNA encoding a CAR to immune such as T cells. CAR expression may be under the control of regulated eukaryotic promoter such as, the CMV promoter, EF1alpha promoter, or Ubiquitin promoter. The vector may also contain a selectable marker to facilitate their manipulation in vitro. In some embodiments, the CAR can be expressed from mRNA in vitro transcribed from a DNA template. These constructs can be made following any number of protocols, such as those taught by Sambrook et al., Molecular Cloning: A Laboratory Manual, 3.sup.rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994, relevant portions incorporated herein by reference.

In some embodiments, the present disclosure provides soluble TCRs. Soluble TCRs are useful, not only for the purpose of investigating specific TCR-peptide-MHC interactions, but as a diagnostic tool to detect cancer, or to detect cancer markers. Soluble TCRs can be used for staining, e.g., to determine the presence of the KRAS G12>V mutation peptide in the context of the MHC-Class II. Soluble TCRs can also be used to deliver a therapeutic agent, for example a cytotoxic compound, to cells presenting the KRAS G12>V mutation peptide.

Adoptive Cell Transfer Therapy. The TCR of the present disclosure can also be used for adoptive cell transfer therapy of immune cells, such as autologous or allogeneic T cells, or even, regulatory T cells, CD4+ T cells, CD8+ T cells, gamma-delta T cells, NK cells, invariant NK cells, NKT cells, mesenchymal stem cell, or pluripotent stem cells) therapy are transfected to express the TCR or binding fragments thereof that bind the KRAS G12>V mutation peptide. Generally, adoptive T cell therapies include genetically engineered TCR-transduced T cells by expressing an alpha chain having at least 90, 95, 98, or 99% identity to the amino acid sequence of SEQ ID NO: 25, 27, 29, 31, 33, 35, 37, 39, 41, 43 and/or a beta chain having at least 90% identity to the amino acid sequence of SEQ ID NO: 26, 28, 30, 32, 34, 36, 38, 40, 42, and 44, or binding fragments of each. The immune cells thus engineered are provided for the treatment of cancer comprising introducing into the subject the engineered cells, such as the engineered T cells. Often, the adoptive cell transfer therapy is provided to a human patient in combination with as second therapy, such as a chemotherapy, a radiotherapy, a surgery, or a second immunotherapy.

The TCR-engineered cells of the present disclosure are provided to a subject as an immunotherapy to target cancer cells. T cells transfected to express the TCR of the present disclosure will often be autologous but can be allogeneic. For making the TCR engineered T cells, autologous T cells are isolated from the patient and are modified to express the TCR of the present disclosure. If the T cells are allogeneic, these are often pooled from several donors or can be T cell clones. The engineered T cells are administered to the subject of interest in an amount sufficient to control, reduce, or eliminate symptoms and signs of the disease being treated.

The isolated T cells can be obtained from blood, bone marrow, lymph, umbilical cord, or lymphoid organs. Generally, the T cells are primary cells, such as those isolated directly from a subject and/or isolated from a subject and frozen. The cells can include one or more subsets of T cells or other cell types, such as whole T cell populations, or isolated subpopulations of T cells, such as CD4+ cells, CD8+ cells, and subpopulations thereof, which can be further divided by function, activation state, maturity, potential for differentiation, expansion, recirculation, localization, presence in a particular organ or compartment, marker or cytokine secretion profile, and/or degree of differentiation.

Sub-types and subpopulations of T cells for use with the present disclosure can be, e.g., CD4+ and/or CD8+ T cells, naive T (T_{N}) cells, effector T cells (T_{EFF}), memory T cells and sub-types thereof, such as stem cell memory T (TSC_{M}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, or follicular helper T cells.

T cells can be pooled and rapidly expanded to provides an increase in the number of antigen-specific T cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days. T cells can be rapidly expanded using non-specific T cell receptor stimulation in the presence of feeder lymphocytes and either interleukin-2 (IL-2) or interleukin-15 (IL-15), non-specific T cell receptor stimulus such as OKT3. T cells can be rapidly expanded by stimulation of peripheral blood mononuclear cells (PBMC) in vitro with one or more antigens in the presence of a T cell growth factor, such as IL-2 or IL-15.

The engineered immune cells of the present disclosure may be administered intravenously, intramuscularly, subcutaneously, transdermally, intraperitoneally, intrathecally, parenterally, intrathecally, intracavitary, intraventricularly, intra-arterially, or via the cerebrospinal fluid, or by any implantable or semi-implantable, permanent or degradable device. The appropriate dosage of the engineered immune cell therapy, such as engineered T cells, may be determined based on the type of disease to be treated, severity and course of the disease, the clinical condition of the individual, the individual's clinical history and response to the treatment, and knowledge and skill of an attending physician.

The engineered immune cells may be made into a pharmaceutical composition or made into an implant appropriate for administration in vivo, with appropriate carriers or diluents, that are pharmaceutically acceptable. The introduction of the cells of the present disclosure can follow the guidance described in the art (see, for instance, Remington's Pharmaceutical Sciences, 16th Ed., Mack, ed. (1980), relevant portions incorporated herein by reference). Generally, transduced T cells expressing a CAR can be formulated into a preparation in liquid or semisolid form. Generally, a pharmaceutically acceptable form is employed that does not kill or reduce the effectiveness of the cells expressing the chimeric receptor. Thus, the engineered T cells can be made into a pharmaceutical composition containing a balanced salt solution such as Hanks' balanced salt solution, or normal saline.

For example, in certain cases, the present disclosure is delivered by intratumoral injection or injection into the vasculature in, or adjacent to, the tumor when targeting discrete, solid, accessible tumors. In other cases, local, regional or systemic administration also may be appropriate. For tumors of >4 cm, the volume to be administered will be about 4-10 ml (in particular 10 ml), while for tumors of <4 cm, a volume of about 1-3 ml will be used (in particular 3 ml). Multiple injections delivered as single dose comprise about 0.1 to about 0.5 ml volumes.

The present disclosure can also be delivered by any number of vectors, liposomes, or even naked DNA to introduce the TCR into host cells, such as host immune cells. Methods of stably transfecting T cells by electroporation using naked DNA are known in the art. Naked DNA generally refers to the DNA encoding a TCR of the present disclosure in a plasmid expression vector under the control of a promoter that drives expression. Alternatively, the present disclosure can be delivered using a viral vector (e.g., a retroviral vector, adenoviral vector, adeno-associated viral vector, or lentiviral vector) that introduces the chimeric construct into T cells. Generally, a vector encoding a CAR that is used for transfecting a T cell from a subject should generally be non-replicating in the subject's T cells. A large number of vectors are known that are based on viruses, where the copy number of the virus maintained in the cell is low enough to maintain viability of the cells, such as, pFB-neo vectors or vectors based on SV40, HIV, HSV, EBV, or BPV.

Nucleic Acids. The present disclosure includes polynucleotides encoding an isolated TCR, CAR, or soluble peptide the TCR comprises an alpha chain having at least 90, 95, 98, or 99% identity to the nucleotide sequence of SEQ ID NO: 49, 51, 53, 55, 57, 59, 61, 63, 65, 67, 69, 71 and/or a beta chain having at least 95% identity to the nucleotide sequence of SEQ ID NO: 50, 52, 54, 56, 58, 60, 62, 64, 66, 68, 70, 72, and 74. The term “nucleic acid” is intended to include DNA and RNA and can be either double stranded or single stranded.

A recombinant expression vector contains one or more of the polynucleotides of the present disclosure, as well as, regulatory sequences selected on the basis of the host cells to be used for expression, to which the one or more polynucleotides are operatively linked. As used herein, the terms “operatively linked” or “operably linked” refer to the one or more polynucleotide(s) linked to regulatory sequences to allow expression of the one or more polynucleotide(s).

The present disclosure includes an engineered T cell receptor (TCR) comprising an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24, wherein the TCR is specific for a KRAS G12>V mutation peptide. In one aspect, the engineered TCR binds to the KRAS G12>V mutation peptide in a complex with HLA DRB5*01:01. In another aspect, the TCR comprises an alpha chain having at least 90, 95, 98, or 99% identity to the amino acid sequence of SEQ ID NO: 25, 27, 29, 31, 33, 35, 37, 39, 41, 43 and/or a beta chain having at least 90% identity to the amino acid sequence of SEQ ID NO: 26, 28, 30, 32, 34, 36, 38, 40, 42, and 44. In another aspect, the TCR is humanized. In another aspect, the TCR comprises an alpha chain having at least 90, 95, 98, or 99% identity to the nucleotide sequence of SEQ ID NO: 49, 51, 53, 55, 57, 59, 61, 63, 65, 67, 69, 71 and/or a beta chain having at least 95% identity to the nucleotide sequence of SEQ ID NO: 50, 52, 54, 56, 58, 60, 62, 64, 66, 68, 70, 72, and 74. In another aspect, the TCR is further defined as a soluble TCR, wherein the soluble TCR does not comprise a transmembrane domain, or comprises transmembrane domain that is a CD28 transmembrane domain or a CD8a transmembrane domain, or further comprises a T-cell signaling domain of any one of the following proteins: a human CD8-alpha protein, a human CD28 protein, a human CD3-zeta protein, a human FcRγ protein, a CD27 protein, an OX40 protein, a human 4-1BB protein, or any combination of the foregoing. In another aspect, the TCR further comprising a detectable label. In another aspect, the TCR is covalently bound to a therapeutic agent, an immunotoxin or a chemotherapeutic agent. In another aspect, the TCR does not recognize wild-type RAS, and the CDR3 is selected from SEQ ID NO: 1, 3, 5 and a beta chain CDR3 having the amino acid sequence of SEQ ID NO: 2, 4, 6. In another aspect, the TCR is part of a multivalent TCR complex comprising a plurality of TCRs according to claim 1. In another aspect, the multivalent TCR comprises 2, 3, 4 or more TCRs associated with one another; wherein the multivalent TCR is present in a lipid bilayer, in a liposome, or is attached to a nanoparticle; or wherein the TCRs are associated with one another via a linker molecule.

The present disclosure includes a polypeptide encoding the TCR comprising an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24, wherein the TCR is specific for a KRAS G12>V mutation peptide.

The present disclosure includes a polynucleotide encoding TCR polypeptide(s) comprising an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24, wherein the TCR is specific for a KRAS G12>V mutation peptide.

The present disclosure includes an expression vector encoding TCR polypeptide(s) comprising an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24, wherein the TCR is specific for a KRAS G12>V mutation peptide. In one aspect, the sequence encoding the TCR is under the control of a promoter. In another aspect, the expression vector is a viral or a retroviral vector. In another aspect, the vector further encodes a linker domain positioned between the alpha chain and beta chain. In another aspect, the linker domain comprises one or more protease cleavage sites, or wherein the one or more cleavage sites are separated by a spacer.

The present disclosure includes a host cell engineered to express a polypeptide encoding the TCR comprising an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24, wherein the TCR is specific for a KRAS G12>V mutation peptide. In another aspect, the cell is a T cell, NK cell, invariant NK cell, NKT cell, mesenchymal stem cell (MSC), or induced pluripotent stem (iPS) cell. In another aspect, the host cell is an immune cell. In another aspect, the T cell is a CD8⁺ T cell, CD4⁺ T cell, or γδ T cell. In another aspect, the T cell is a regulatory T cell (Treg). In another aspect, the host cell is autologous or allogeneic.

The present disclosure includes a method for engineering a host cell comprising contacting an immune cell with the TCR or the expression vector of the present disclosure. In one aspect, the method comprises contacting is further defined as transfecting or transducing, wherein transfecting comprises electroporating RNA encoding the TCR described hereinabove into the immune cell.

The present disclosure includes a method for treating a subject with a cancer comprising a KRAS G12>V mutation peptide, the method comprising: administering to the subject an effective amount of one or more immune cells modified by cloning genes of the alpha and beta chains of a T cell receptor (TCR) ex vivo to express a chimeric antigen receptor specific for the KRAS G12>V mutation, wherein the chimeric antigen receptor comprises an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24. In one aspect, the immune cell is T cell, NK cell, invariant NK cell, NKT cell, mesenchymal stem cell (MSC), or induced pluripotent stem (iPS) cell, or a peripheral blood lymphocyte. In another aspect, method further comprises at least one of: sorting the immune cells into T cells to isolate TCR engineered T cells; performing a T cell cloning of the immune cells by serial dilution; or expanding a T cell clone from the immune cells by a rapid expansion protocol. In another aspect, the subject is identified to have an HLA DRB5*01:01 allele. In another aspect, the immune cell is a T cell selected from a CD8⁺ T cell, CD4⁺ T cell, or Treg. In another aspect, the cancer is selected from colorectal cancer, pancreatic cancer, renal cancer, lung cancer, liver cancer, breast cancer, prostate cancer, gastrointestinal cancer, peritoneal cancer, melanoma, endometrial cancer, ovarian cancer, cervical cancer, uterine carcinoma, bladder cancer, glioblastoma, brain metastases, salivary gland carcinoma, thyroid cancer, brain cancer, lymphoma, myeloma, and head and neck cancer. In another aspect, the cancer is selected from pancreatic ductal adenocarcinoma and colorectal adenocarcinoma. In another aspect, the TCR engineered cells are autologous or allogeneic. In another aspect, the method further comprises administering a second anticancer selected from chemotherapy, immunotherapy, surgery, radiotherapy, or biotherapy. In another aspect, the one or more immune cells are administered intravenously, intraperitoneally, intratracheally, intratumorally, intramuscularly, endoscopically, intralesionally, percutaneously, subcutaneously, regionally, or by direct injection or perfusion.

The present disclosure includes chimeric antigen receptor expressing T cell (CAR-T) comprising an antigen recognition moiety and a T-cell activation moiety, wherein the T-cell activation moiety comprises a transmembrane domain, and wherein the antigen recognition moiety is directed against a KRAS G12>V mutation. In another aspect, the antigen recognition moiety does not recognize non-mutated RAS. In another aspect, the transmembrane domain is a CD28 transmembrane domain or a CD8a transmembrane domain. In another aspect, the T-cell activation moiety comprises a T-cell signaling domain of any one of the following proteins: a human CD8-alpha protein, a human CD28 protein, a human CD3-zeta protein, a human FcRγ protein, a CD27 protein, an OX40 protein, a human 4-1BB protein, or any combination of the foregoing. In another aspect, the antigen recognition moiety comprises the amino acid sequence of wherein the TCR comprises an alpha chain variable region having at least 90, 95, 98, or 99% identity to the amino acid sequence of SEQ ID NO: 25, 27, 29, 31, 33, 35, 37, 39, 41, 43 and/or a beta chain variable region having at least 90% identity to the amino acid sequence of SEQ ID NO: 26, 28, 30, 32, 34, 36, 38, 40, 42, and 44. In another aspect, the antigen recognition moiety comprises an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24.

### Example 1

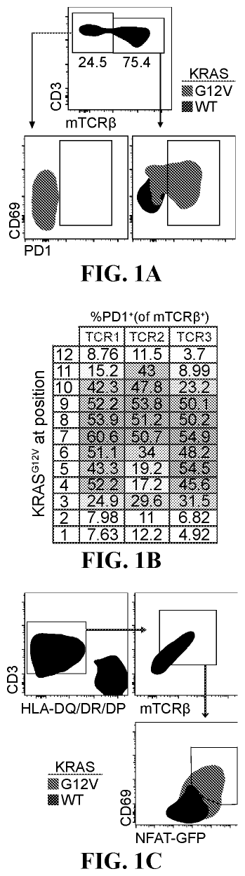

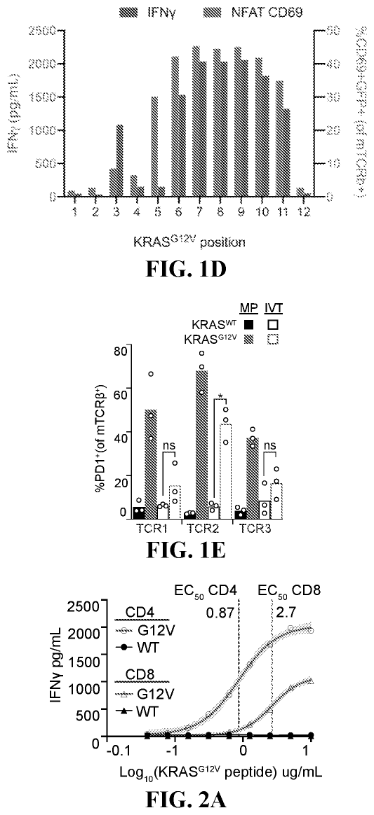

FIGS. 1A to 1D shows that three novel KRAS^{G12V}-reactive TCRs recognize distinct mutant epitopes, but only TCR2 recognizes processed and presented neoantigen. Candidate human TCRαβ pairs and corresponding V(D)J sequences were linked to murine TCR constant (mTCR) regions and cloned into the MSGV1 retroviral vector. Primary healthy donor (HD) CD4 T cells and TCR-deficient J76 cells were transduced with retrovirus containing these novel KRAS^{G12V}-reactive TCRs. Patient-derived EBV transformed B cell lines were established for cocultures. T cells and B cells were cocultured 1:1 overnight (ON) with indicated conditions. (FIG. 1A) Gating strategy for HD CD4 T cells used to assess TCR reactivity. Prior gating not shown: Singlets, DAPI⁻CD4⁺. (FIG. 1B) B cells were pulsed ON with 10 ug/mL of 15mer minimal peptides (MP) containing the KRAS^{G12V} mutation at various positions. Prior to coculture, B cells were washed and resuspended 1:1 with transduced CD4 T cells. PD1 upregulation was assessed the following day according to the strategy in FIG. 1A. (FIG. 1C) Gating strategy for TCR transduced J76 cells containing an NFAT-GFP reporter. (FIG. 1D) Supernatants from TCR2 in FIG. 1B were probed for IFNγ (dark gray) and percent GFP⁺CD69⁺ J76 cells (light gray) as in C for TCR2. (E) B cells were either pulsed with MP or electroporated with IVT mRNA containing the KRAS^{G12V} mutation prior to ON coculture.

Table 1. HLA genotypes of various EBV B cell lines that can (PT-66, PT-37, D66, D8, GM3107) and cannot (PT-159, PT-198) trigger TCR2. KRAS^{G12V}-specific TCRs were identified from PT-66.

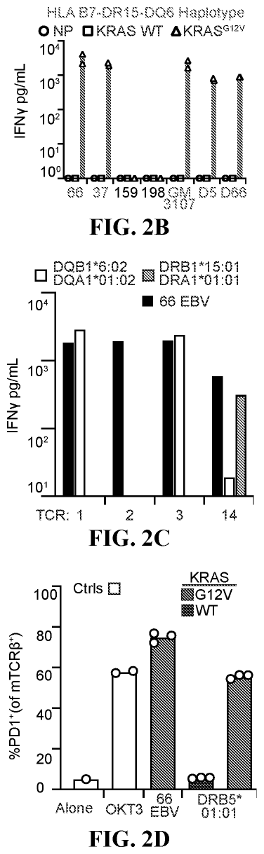

FIGS. 2A to 2D shows the coreceptor dependence, avidity, and restriction characteristics for TCR2. (FIG. 2A) Both HD CD4 and CD8 T cells were transduced with TCR2 and cocultured B cells with decreasing amounts of KRAS^{G12V} minimal peptide. IFNγ production was assessed from the supernatants the following day. (FIG. 2B) B cells that match certain HLA alleles as shown in Table 1 were pulsed with indicated peptides and cocultured with TCR2 transduced CD4 T cells. IFNγ production was assessed from the supernatants the following day. (FIG. 2C) 293T cells transduced with indicated MHCII HLA heterodimers or PT66's EBV transformed B cell line (LCL) were pulsed with KRAS^{G12V}MP and cocultured with transduced CD4 T cells. IFNγ production was assessed from the supernatants the following day. (FIG. 2D) Stably transfected 293T cells with DRB5*01:01 were pulsed with WT or mutant MP and subsequently cocultured with TCR2 transduced HD CD4 T cells. Activation by surface PD1 expression was assessed flow cytometrically the following day.

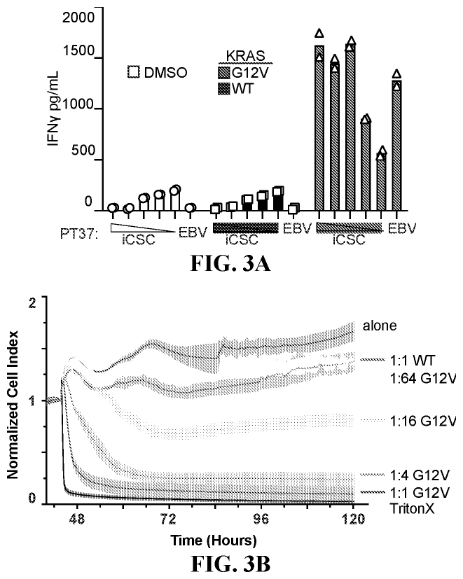

FIGS. 3A and 3 shows that TCR2 recognizes and kills tumor cells directly. An inducible cancer stem cell (iCSC) from PT37 which matches PT66 restriction requirements for TCR2, but lacks the KRAS^{G12V} mutation. (FIG. 3A) iCSCs were treated with 10 ng/mL of IFNγ ON to induce MHCII expression and then pulsed with indicated MP ON. 48 hours after treatment, HD CD4 T cells transduced with TCR2 were added to the culture with decreasing numbers of target iCSCs. 100,000 iCSC with 1:1 dilution to 6,250 iCSC. 100,000 MP-pulsed EBV B cells were used as controls. IFNγ production was assessed from the supernatants the following day. (FIG. 3B) 25,000 PT37 iCSCs were seeded in the ACEA xCelligence platform and treated as in FIG. 3A. Various titrations of TCR2-transduced HD CD4 T cells were added and growth kinetics assessed over 3 days with mutant and WT MP pulsed iCSCs. Triton-X (lower black curve) served as full lysis control, and untreated (upper black curve, “alone”) serves as no treatment.

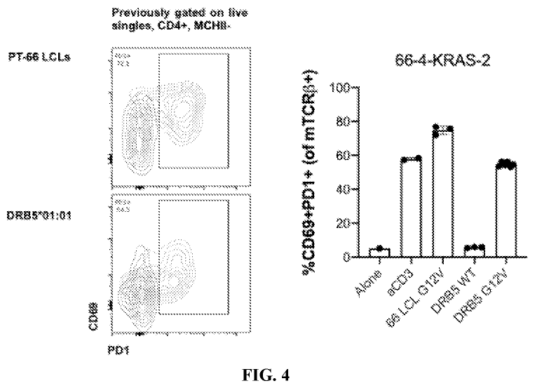

FIG. 4 shows the gating of cell, and the MHC restriction of the T cell receptors of the present disclosure. For TCR2 it was found that it was restricted to HLA DR1B5*01:01. DRB5*01:01 was transfected into PT-66 LCL cells for recognition by TCR2. For TCRs (TCR1 and TCR3) the G12>V peptide triggered a T cell response in the context of DQ6.2 (DQA1*0102:DQB1*0602).

TABLE 2
CDR3 amino acid sequences,
TCR alpha and TCR beta

| | | | SEQ |
|---|---|---|---|
| | | | ID |
| Name | TCR Chain | AA Seq CDR | NO: |
| 66-4-KRAS-1 | ALPHA: | CAVSVGPGNTGKLIF | 1 |
| | BETA: | CASSLGVLGLRYF | 2 |
| 66-4-KRAS-2 | ALPHA: | CAENSGGSNYKLTF | 3 |
| | BETA: | CASSWERAGKAFF | 4 |
| 66-4-KRAS-3 | ALPHA: | CAMSVFIYSTFIF | 5 |
| | BETA: | CASSGRQETQYF | 6 |
| 66-4-KRAS-6 | ALPHA: | CATDAYTRQLTF | 7 |
| | BETA: | CASGGPGANRPQHF | 8 |
| 66-4-KRAS-8 | ALPHA: | CAYRSLWGSGYALNF | 9 |
| | BETA: | CSVEKGAQETQYF | 10 |
| 66-4-KRAS-11 | ALPHA: | CAALFGNEKLTF | 11 |
| | BETA: | CATLQGWSYNEQFF | 12 |
| 66-4-KRAS-12 | ALPHA: | CALSTGGFKTIF | 13 |
| | BETA: | CSVLGPGTGGRGAN | 14 |
| | | YGYTF | |
| 66-4-KRAS-13 | ALPHA: | CATTIDGQKLLF | 15 |
| | BETA: | CSASDRGSGELFF | 16 |
| 66-4-KRAS-14 | ALPHA: | CATDAYNARLMF | 17 |
| | BETA: | CSVVASGSVDTQYF | 18 |
| 66-4-KRAS-19 | ALPHA: | CAGPAGAQKLVF | 19 |
| | BETA: | CASSPVPYSGNTIYF | 20 |
| 66-4-KRAS-22 | ALPHA: | CAVSKSDKIIF | 21 |
| | BETA: | CATSEGGSTGTEAFF | 22 |
| 66-4-KRAS-25 | ALPHA: | CALNRNTGNQFYF | 23 |
| | BETA: | CASSIPQGNGYTF | 24 |

TABLE 3
Full length amino acid sequences,
TCR alpha and TCR beta

| | | | SEQ |
|---|---|---|---|
| | TCR | | ID |
| | Chain | AA Sequence | NO: |
| 66-4- | ALPHA: | MLLLLVPVLEVIFTLGGTRAQSVTQLGSHV | 25 |
| KRAS-1 | | SVSEGALVLLRCNYSSSVPPYLFWYVQYPN | |
| | | QGLQLLLKYTSAATLVKGINGFEAEFKKSE | |
| | | TSFHLTKPSAHMSDAAEYFCAVSVGPGNTG | |
| | | KLIFGQGTTLQVKPDIQNPDPAVYQLRDSK | |
| | | SSDKSVCLFTDFDSQTNVSQSKDSDVYITD | |
| | | KTVLDMRSMDFKSNSAVAWSNKSDFACANA | |
| | | FNNSIIPEDTFFPSPESSCDVKLVEKSFET | |
| | | DTNLNFQNLSVIGFRILLLKVAGFNLLMTL | |
| | | RLWSS* | |
| | BETA: | MGIRLLCRVAFCFLAVGLVDVKVTQSSRYL | 26 |
| | | VKRTGEKVFLECVQDMDHENMFWYRQDPGL | |
| | | GLRLIYFSYDVKMKEKGDIPEGYSVSREKK | |
| | | ERFSLILESASTNQTSMYLCASSLGVLGLR | |
| | | YFGPGTRLTVTEDLNKVFPPEVAVFEPSEA | |
| | | EISHTQKATLVCLATGFFPDHVELSWWVNG | |
| | | KEVHSGVSTDPQPLKEQPALNDSRYCLSSR | |
| | | LRVSATFWQNPRNHFRCQVQFYGLSENDEW | |
| | | TQDRAKPVTQIVSAEAWGRADCGFTSVSYQ | |
| | | QGVLSATILYEILLGKATLYAVLVSALVLM | |
| | | AMVKRKDF* | |
| 66-4- | ALPHA: | MAGIRALFMYLWLQLDWVSRGESVGLHLPT | 27 |
| KRAS-2 | | LSVQEGDNSIINCAYSNSASDYFIWYKQES | |
| | | GKGPQFIIDIRSNMDKRQGQRVTVLLNKTV | |
| | | KHLSLQIAATQPGDSAVYFCAENSGGSNYK | |
| | | LTFGKGTLLTVNPNIQNPDPAVYQLRDSKS | |
| | | SDKSVCLFTDFDSQTNVSQSKDSDVYITDK | |
| | | TVLDMRSMDFKSNSAVAWSNKSDFACANAF | |
| | | NNSIIPEDTFFPSPESSCDVKLVEKSFETD | |
| | | TNLNFQNLSVIGFRILLLKVAGFNLLMTLR | |
| | | LWSS* | |
| | BETA: | MGPGLLCWVLLCLLGAGSVETGVTQSPTHL | 28 |
| | | IKTRGQQVTLRCSSQSGHNTVSWYQQALGQ | |
| | | GPQFIFQYYREEENGRGNFPPRFSGLQFPN | |
| | | YSSELNVNALELDDSALYLCASSWERAGKA | |
| | | FFGQGTRLTVVEDLNKVFPPEVAVFEPSEA | |
| | | EISHTQKATLVCLATGFFPDHVELSWWVNG | |
| | | KEVHSGVSTDPQPLKEQPALNDSRYCLSSR | |
| | | LRVSATFWQNPRNHFRCQVQFYGLSENDEW | |
| | | TQDRAKPVTQIVSAEAWGRADCGFTSVSYQ | |
| | | QGVLSATILYEILLGKATLYAVLVSALVLM | |
| | | AMVKRKDF* | |
| 66-4- | ALPHA: | MMKSLRVLLVILWLQLSWVWSQQKEVEQDP | 29 |
| KRAS-3 | | GPLSVPEGAIVSLNCTYSNSAFQYFMWYRQ | |
| | | YSRKGPELLMYTYSSGNKEDGRFTAQVDKS | |
| | | SKYISLFIRDSQPSDSATYLCAMSVFIYST | |
| | | FIFGSGTRLSVKPDIQNPDPAVYQLRDSKS | |
| | | SDKSVCLFTDFDSQTNVSQSKDSDVYITDK | |
| | | TVLDMRSMDFKSNSAVAWSNKSDFACANAF | |
| | | NNSIIPEDTFFPSPESSCDVKLVEKSFETD | |
| | | TNLNFQNLSVIGFRILLLKVAGFNLLMTLR | |
| | | LWSS* | |
| | BETA: | MGIRLLCRVAFCFLAVGLVDVKVTQSSRYL | 30 |
| | | VKRTGEKVFLECVQDMDHENMFWYRQDPGL | |
| | | GLRLIYFSYDVKMKEKGDIPEGYSVSREKK | |
| | | ERFSLILESASTNQTSMYLCASSGRQETQY | |
| | | FGPGTRLLVLEDLNKVFPPEVAVFEPSEAE | |
| | | ISHTQKATLVCLATGFFPDHVELSWWVNGK | |
| | | EVHSGVSTDPQPLKEQPALNDSRYCLSSRL | |
| | | RVSATFWQNPRNHFRCQVQFYGLSENDEWT | |
| | | QDRAKPVTQIVSAEAWGRADCGFTSVSYQQ | |
| | | GVLSATILYEILLGKATLYAVLVSALVLMA | |
| | | MVKRKDF* | |
| 66-4- | ALPHA: | METLLGVSLVILWLQLARVNSQQGEEDPQA | 31 |
| KRAS-6 | | LSIQEGENATMNCSYKTSINNLQWYRQNSG | |
| | | RGLVHLILIRSNEREKHSGRLRVTLDTSKK | |
| | | SSSLLITASRAADTASYFCATDAYTRQLTF | |
| | | GSGTQLTVLPDIQNPDPAVYQLRDSKSSDK | |
| | | SVCLFTDFDSQTNVSQSKDSDVYITDKTVL | |
| | | DMRSMDFKSNSAVAWSNKSDFACANAFNNS | |
| | | IIPEDTFFPSPESSCDVKLVEKSFETDTNL | |
| | | NFQNLSVIGFRILLLKVAGFNLLMTLRLWS | |
| | | S* | |
| | BETA: | MSNQVLCCVVLCLLGANTVDGGITQSPKYL | 32 |
| | | FRKEGQNVTLSCEQNLNHDAMYWYRQDPGQ | |
| | | GLRLIYYSQIVNDFQKGDIAEGYSVSREKK | |
| | | ESFPLTVTSAQKNPTAFYLCASGGPGANRP | |
| | | QHFGDGTRLSILEDLNKVFPPEVAVFEPSE | |
| | | AEISHTQKATLVCLATGFFPDHVELSWWVN | |
| | | GKEVHSGVSTDPQPLKEQPALNDSRYCLSS | |
| | | RLRVSATFWQNPRNHFRCQVQFYGLSENDE | |
| | | WTQDRAKPVTQIVSAEAWGRADCGFTSVSY | |
| | | QQGVLSATILYEILLGKATLYAVLVSALVL | |
| | | MAMVKRKDF* | |
| 66-4- | ALPHA: | MACPGFLWALVISTCLEFSMAQTVTQSQPE | 33 |
| KRAS-8 | | MSVQEAETVTLSCTYDTSESDYYLFWYKQP | |
| | | PSRQMILVIRQEAYKQQNATENRFSVNFQK | |
| | | AAKSFSLKISDSQLGDAAMYFCAYRSLWGS | |
| | | GYALNFGKGTSLLVTPHIQNPDPAVYQLRD | |
| | | SKSSDKSVCLFTDFDSQTNVSQSKDSDVYI | |
| | | TDKTVLDMRSMDFKSNSAVAWSNKSDFACA | |
| | | NAFNNSIIPEDTFFPSPESSCDVKLVEKSF | |
| | | ETDTNLNFQNLSVIGFRILLLKVAGFNLLM | |
| | | TLRLWSS* | |
| | BETA: | MLSLLLLLLGLGSVFSAVISQKPSRDICQR | 34 |
| | | GTSLTIQCQVDSQVTMMFWYRQQPGQSLTL | |
| | | IATANQGSEATYESGFVIDKFPISRPNLTF | |
| | | STLTVSNMSPEDSSIYLCSVEKGAQETQYF | |
| | | GPGTRLLVLEDLNKVFPPEVAVFEPSEAEI | |
| | | SHTQKATLVCLATGFFPDHVELSWWVNGKE | |
| | | VHSGVSTDPQPLKEQPALNDSRYCLSSRLR | |
| | | VSATFWQNPRNHFRCQVQFYGLSENDEWTQ | |
| | | DRAKPVTQIVSAEAWGRADCGFTSVSYQQG | |
| | | VLSATILYEILLGKATLYAVLVSALVLMAM | |
| | | VKRKDF* | |
| 66-4- | ALPHA: | MTSIRAVFIFLWLQLDLVNGENVEQHPSTL | 35 |
| KRAS-11 | | SVQEGDSAVIKCTYSDSASNYFPWYKQELG | |
| | | KGPQLIIDIRSNVGEKKDQRIAVTLNKTAK | |
| | | HFSLHITETQPEDSAVYFCAALFGNEKLTF | |
| | | GTGTRLTIIPNIQNPDPAVYQLRDSKSSDK | |
| | | SVCLFTDFDSQTNVSQSKDSDVYITDKTVL | |
| | | DMRSMDFKSNSAVAWSNKSDFACANAFNNS | |
| | | IIPEDTFFPSPESSCDVKLVEKSFETDTNL | |
| | | NFQNLSVIGFRILLLKVAGFNLLMTLRLWS | |
| | | S* | |
| | BETA: | MGTRLLFWVAFCLLGADHTGAGVSQSPSNK | 36 |
| | | VTEKGKDVELRCDPISGHTALYWYRQSLGQ | |
| | | GLEFLIYFQGNSAPDKSGLPSDRFSAERTG | |
| | | GSVSTLTIQRTQQEDSAVYLCATLQGWSYN | |
| | | EQFFGPGTRLTVLEDLNKVFPPEVAVFEPS | |
| | | EAEISHTQKATLVCLATGFFPDHVELSWWV | |
| | | NGKEVHSGVSTDPQPLKEQPALNDSRYCLS | |
| | | SRLRVSATFWQNPRNHFRCQVQFYGLSEND | |
| | | EWTQDRAKPVTQIVSAEAWGRADCGFTSVS | |
| | | YQQGVLSATILYEILLGKATLYAVLVSALV | |
| | | LMAMVKRKDF* | |
| 66-4- | ALPHA: | MKPTLISVLVIIFILRGTRAQRVTQPEKLL | 37 |
| KRAS-12 | | SVFKGAPVELKCNYSYSGSPELFWYVQYSR | |
| | | QRLQLLLRHISRESIKGFTADLNKGETSFH | |
| | | LKKPFAQEEDSAMYYCALSTGGFKTIFGAG | |
| | | TRLFVKANIQNPDPAVYQLRDSKSSDKSVC | |
| | | LFTDFDSQTNVSQSKDSDVYITDKTVLDMR | |
| | | SMDFKSNSAVAWSNKSDFACANAFNNSIIP | |
| | | EDTFFPSPESSCDVKLVEKSFETDTNLNFQ | |
| | | NLSVIGFRILLLKVAGFNLLMTLRLWSS* | |
| | BETA: | MLSLLLLLLGLGSVFSAVISQKPSRDICQR | 38 |
| | | GTSLTIQCQVDSQVTMMFWYRQQPGQSL | |
| | | TLIATANQGSEATYESGFVIDKFPISRPNL | |
| | | TFSTLTVSNMSPEDSSIYLCSVLGPGTGGR | |
| | | GANYGYTFGSGTRLTVVEDLNKVFPPEVAV | |
| | | FEPSEAEISHTQKATLVCLATGFFPDHVEL | |
| | | SWWVNGKEVHSGVSTDPQPLKEQPALNDSR | |
| | | YCLSSRLRVSATFWQNPRNHFRCQVQFYGL | |
| | | SENDEWTQDRAKPVTQIVSAEAWGRADCGF | |
| | | TSVSYQQGVLSATILYEILLGKATLYAVLV | |
| | | SALVLMAMVKRKDF* | |
| 66-4- | ALPHA: | METLLGVSLVILWLQLARVNSQQGEEDPQA | 39 |
| KRAS-13 | | LSIQEGENATMNCSYKTSINNLQWYRQNSG | |
| | | RGLVHLILIRSNEREKHSGRLRVTLDTSKK | |
| | | SSSLLITASRAADTASYFCATTIDGQKLLF | |
| | | ARGTMLKVDLNIQNPDPAVYQLRDSKSSDK | |
| | | SVCLFTDFDSQTNVSQSKDSDVYITDKTVL | |
| | | DMRSMDFKSNSAVAWSNKSDFACANAFNNS | |
| | | IIPEDTFFPSPESSCDVKLVEKSFETDTNL | |
| | | NFQNLSVIGFRILLLKVAGFNLLMTLRLWS | |
| | | S* | |
| | BETA: | MLLLLLLLGPAGSGLGAVVSQHPSRVICKS | 40 |
| | | GTSVKIECRSLDFQATTMFWYRQFPKKSLM | |
| | | LMATSNEGSKATYEQGVEKDKFLINHASLT | |
| | | LSTLTVTSAHPEDSSFYICSASDRGSGELF | |
| | | FGEGSRLTVLEDLNKVFPPEVAVFEPSEAE | |
| | | ISHTQKATLVCLATGFFPDHVELSWWVNGK | |
| | | EVHSGVSTDPQPLKEQPALNDSRYCLSSRL | |
| | | RVSATFWQNPRNHFRCQVQFYGLSENDEWT | |
| | | QDRAKPVTQIVSAEAWGRADCGFTSVSYQQ | |
| | | GVLSATILYEILLGKATLYAVLVSALVLMA | |
| | | MVKRKDF* | |
| 66-4- | ALPHA: | METLLGVSLVILWLQLARVNSQQGEEDPQA | 41 |
| KRAS-14 | | LSIQEGENATMNCSYKTSINNLQWYRQNSG | |
| | | RGLVHLILIRSNEREKHSGRLRVTLDTSKK | |
| | | SSSLLITASRAADTASYFCATDAYNARLMF | |
| | | GDGTQLVVKPNIQNPDPAVYQLRDSKSSDK | |
| | | SVCLFTDFDSQTNVSQSKDSDVYITDKTVL | |
| | | DMRSMDFKSNSAVAWSNKSDFACANAFNNS | |
| | | IIPEDTFFPSPESSCDVKLVEKSFETDTNL | |
| | | NFQNLSVIGFRILLLKVAGFNLLMTLRLWS | |
| | | S* | |
| | BETA: | MLSLLLLLLGLGSVFSAVISQKPSRDICQR | 42 |
| | | GTSLTIQCQVDSQVTMMFWYRQQPGQSLTL | |
| | | IATANQGSEATYESGFVIDKFPISRPNLTF | |
| | | STLTVSNMSPEDSSIYLCSVVASGSVDTQY | |
| | | FGPGTRLTVLEDLNKVFPPEVAVFEPSEAE | |
| | | ISHTQKATLVCLATGFFPDHVELSWWVNGK | |
| | | EVHSGVSTDPQPLKEQPALNDSRYCLSSRL | |
| | | RVSATFWQNPRNHFRCQVQFYGLSENDEWT | |
| | | QDRAKPVTQIVSAEAWGRADCGFTSVSYQQ | |
| | | GVLSATILYEILLGKATLYAVLVSALVLMA | |
| | | MVKRKDF* | |
| 66-4- | ALPHA: | MVLKFSVSILWIQLAWVSTQLLEQSPQFLS | 43 |
| KRAS-19 | | IQEGENLTVYCNSSSVFSSLQWYRQEPGEG | |
| | | PVLLVTVVTGGEVKKLKRLTFQFGDARKDS | |
| | | SLHITAAQPGDTGLYLCAGPAGAQKLVFGQ | |
| | | GTRLTINPNIQNPDPAVYQLRDSKSSDKSV | |
| | | CLFTDFDSQTNVSQSKDSDVYITDKTVLDM | |
| | | RSMDFKSNSAVAWSNKSDFACANAFNNSII | |
| | | PEDTFFPSPESSCDVKLVEKSFETDTNLNF | |
| | | QNLSVIGFRILLLKVAGFNLLMTLRLWSS* | |
| | BETA: | MSISLLCCAAFPLLWAGPVNAGVTQTPKFR | 44 |
| | | ILKIGQSMTLQCTQDMNHNYMYWYRQDPGM | |
| | | GLKLIYYSVGAGITDKGEVPNGYNVSRSTT | |
| | | EDFPLRLELAAPSQTSVYFCASSPVPYSGN | |
| | | TIYFGEGSWLTVVEDLNKVFPPEVAVFEPS | |
| | | EAEISHTQKATLVCLATGFFPDHVELSWWV | |
| | | NGKEVHSGVSTDPQPLKEQPALNDSRYCLS | |
| | | SRLRVSATFWQNPRNHFRCQVQFYGLSEND | |
| | | EWTQDRAKPVTQIVSAEAWGRADCGFTSVS | |
| | | YQQGVLSATILYEILLGKATLYAVLVSALV | |
| | | LMAMVKRKDF* | |
| 66-4- | ALPHA: | MLLLLVPVLEVIFTLGGTRAQSVTQLGSHV | 45 |
| KRAS | | SVSEGALVLLRCNYSSSVPPYLFWYVQYPN | |
| -22 | | QGLQLLLKYTSAATLVKGINGFEAEFKKSE | |
| | | TSFHLTKPSAHMSDAAEYFCAVSKSDKIIF | |
| | | GKGTRLHILPNIQNPDPAVYQLRDSKSSDK | |
| | | SVCLFTDFDSQTNVSQSKDSDVYITDKTVL | |
| | | DMRSMDFKSNSAVAWSNKSDFACANAFNNS | |
| | | IIPEDTFFPSPESSCDVKLVEKSFETDTNL | |
| | | NFQNLSVIGFRILLLKVAGFNLLMTLRLWS | |
| | | S* | |
| | BETA: | MASLLFFCGAFYLLGTGSMDADVTQTPRNR | 46 |
| | | ITKTGKRIMLECSQTKGHDRMYWYRQDPGL | |
| | | GLRLIYYSFDVKDINKGEISDGYSVSRQAQ | |
| | | AKFSLSLESAIPNQTALYFCATSEGGSTGT | |
| | | EAFFGQGTRLTVVEDLNKVFPPEVAVFEPS | |
| | | EAEISHTQKATLVCLATGFFPDHVELSWWV | |
| | | NGKEVHSGVSTDPQPLKEQPALNDSRYCLS | |
| | | SRLRVSATFWQNPRNHFRCQVQFYGLSEND | |
| | | EWTQDRAKPVTQIVSAEAWGRADCGFTSVS | |
| | | YQQGVLSATILYEILLGKATLYAVLVSALV | |
| | | LMAMVKRKDF* | |
| 66-4- | ALPHA: | MNYSPGLVSLILLLLGRTRGNSVTQMEGPV | 47 |
| KRAS-25 | | TLSEEAFLTINCTYTATGYPSLFWYVQYPG | |
| | | EGLQLLLKATKADDKGSNKGFEATYRKETT | |
| | | SFHLEKGSVQVSDSAVYFCALNRNTGNQFY | |
| | | FGTGTSLTVIPNIQNPDPAVYQLRDSKSSD | |
| | | KSVCLFTDFDSQTNVSQSKDSDVYITDKTV | |
| | | LDMRSMDFKSNSAVAWSNKSDFACANAFNN | |
| | | SIIPEDTFFPSPESSCDVKLVEKSFETDTN | |
| | | LNFQNLSVIGFRILLLKVAGFNLLMTLRLW | |
| | | SS* | |
| | BETA: | MSNQVLCCVVLCLLGANTVDGGITQSPKYL | 48 |
| | | FRKEGQNVTLSCEQNLNHDAMYWYRQDPGQ | |
| | | GLRLIYYSQIVNDFQKGDIAEGYSVSREKK | |
| | | ESFPLTVTSAQKNPTAFYLCASSIPQGNGY | |
| | | TFGSGTRLTVVEDLNKVFPPEVAVFEPSEA | |
| | | EISHTQKATLVCLATGFFPDHVELSWWVNG | |
| | | KEVHSGVSTDPQPLKEQPALNDSRYCLSSR | |
| | | LRVSATFWQNPRNHFRCQVQFYGLSENDEW | |
| | | TQDRAKPVTQIVSAEAWGRADCGFTSVSYQ | |
| | | QGVLSATILYEILLGKATLYAVLVSALVLM | |
| | | AMVKRKDF* | |

TABLE 4
Full length nucleic acid sequences,
TCR alpha and TCR beta

| | | | SEQ |
|---|---|---|---|
| | | | ID |
| | | NT FULL TCR | NO: |
| 66-4- | ALPHA: | Atgctcctgctgctcgtcccagtgctcgag | 49 |
| KRAS-1 | | gtgatttttaccctgggaggaaccagagcc | |
| | | cagtcggtgacccagcttggcagccacgtc | |
| | | tctgtctctgaaggagccctggttctgctg | |
| | | aggtgcaactactcatcgtctgttccacca | |
| | | tatctcttctggtatgtgcaataccccaac | |
| | | caaggactccagcttctcctgaagtacaca | |
| | | tcagcggccaccctggttaaaggcatcaac | |
| | | ggttttgaggctgaatttaagaagagtgaa | |
| | | acctccttccacctgacgaaaccctcagcc | |
| | | catatgagcgacgcggctgagtacttctgt | |
| | | gctgtgagtgtaggtcctggcaacacaggc | |
| | | aaactaatctttgggcaagggacaacttta | |
| | | caagtaaaaccagatatccagaaccctgac | |
| | | cctgccgtgtaccagctgagagactctaaa | |
| | | tccagtgacaagtctgtctgcctattcacc | |
| | | gattttgattctcaaacaaatgtgtcacaa | |
| | | agtaaggattctgatgtgtatatcacagac | |
| | | aaaactgtgctagacatgaggtctatggac | |
| | | ttcaagagcaacagtgctgtggcctggagc | |
| | | aacaaatctgactttgcatgtgcaaacgcc | |
| | | ttcaacaacagcattattccagaagacacc | |
| | | ttcttccccagcccagaaagttcctgtgat | |
| | | gtcaagctggtcgagaaaagctttgaaaca | |
| | | gatacgaacctaaactttcaaaacctgtca | |
| | | gtgattgggttccgaatcctcctcctgaaa | |
| | | gtggccgggtttaatctgctcatgacgctg | |
| | | cggctgtggtccagcTAA | |
| | BETA: | Atgggaatcaggctcctgtgtcgtgtggcc | 50 |
| | | ttttgtttcctggctgtaggcctcgtagat | |
| | | gtgaaagtaacccagagctcgagatatcta | |
| | | gtcaaaaggacgggagagaaagtttttctg | |
| | | gaatgtgtccaggatatggaccatgaaaat | |
| | | atgttctggtatcgacaagacccaggtctg | |
| | | gggctacggctgatctatttctcatatgat | |
| | | gttaaaatgaaagaaaaaggagatattcct | |
| | | gaggggtacagtgtctctagagagaagaag | |
| | | gagcgcttctccctgattctggagtccgcc | |
| | | agcaccaaccagacatctatgtacctctgt | |
| | | gccagcagtttgggggttctaggcctgcgg | |
| | | tacttcgggccgggcaccaggctcacggtc | |
| | | acagaggacctgaacaaggtgttcccaccc | |
| | | gaggtcgctgtgtttgagccatcagaagca | |
| | | gagatctcccacacccaaaaggccacactg | |
| | | gtgtgcctggccacaggcttcttccctgac | |
| | | cacgtggagctgagctggtgggtgaatggg | |
| | | aaggaggtgcacagtggggtcagcacggac | |
| | | ccgcagcccctcaaggagcagcccgccctc | |
| | | aatgactccagatactgcctgagcagccgc | |
| | | ctgagggtctcggccaccttctggcagaac | |
| | | ccccgcaaccacttccgctgtcaagtccag | |
| | | ttctacgggctctcggagaatgacgagtgg | |
| | | acccaggatagggccaaacccgtcacccag | |
| | | atcgtcagcgccgaggcctggggtagagca | |
| | | gactgtggctttacctcggtgtcctaccag | |
| | | caaggggtcctgtctgccaccatcctctat | |
| | | gagatcctgctagggaaggccaccctgtat | |
| | | gctgtgctggtcagcgcccttgtgttgatg | |
| | | gccatggtcaagagaaaggatttcTAA | |
| 66-4- | ALPHA: | Atggcaggcattcgagctttatttatgtac | 51 |
| KRAS-2 | | ttgtggctgcagctggactgggtgagcaga | |
| | | ggagagagtgtggggctgcatcttcctacc | |
| | | ctgagtgtccaggagggtgacaactctatt | |
| | | atcaactgtgcttattcaaacagcgcctca | |
| | | gactacttcatttggtacaagcaagaatct | |
| | | ggaaaaggtcctcaattcattatagacatt | |
| | | cgttcaaatatggacaaaaggcaaggccaa | |
| | | agagtcaccgttttattgaataagacagtg | |
| | | aaacatctctctctgcaaattgcagctact | |
| | | caacctggagactcagctgtctacttttgt | |
| | | gcagagaatagtggaggtagcaactataaa | |
| | | ctgacatttggaaaaggaactctcttaacc | |
| | | gtgaatccaaatatccagaaccctgaccct | |
| | | gccgtgtaccagctgagagactctaaatcc | |
| | | agtgacaagtctgtctgcctattcaccgat | |
| | | tttgattctcaaacaaatgtgtcacaaagt | |
| | | aaggattctgatgtgtatatcacagacaaa | |
| | | actgtgctagacatgaggtctatggacttc | |
| | | aagagcaacagtgctgtggcctggagcaac | |
| | | aaatctgactttgcatgtgcaaacgccttc | |
| | | aacaacagcattattccagaagacaccttc | |
| | | ttccccagcccagaaagttcctgtgatgtc | |
| | | aagctggtcgagaaaagctttgaaacagat | |
| | | acgaacctaaactttcaaaacctgtcagtg | |
| | | attgggttccgaatcctcctcctgaaagtg | |
| | | gccgggtttaatctgctcatgacgctgcgg | |
| | | ctgtggtccagcTAA | |
| | BETA: | Atgggccctgggctcctctgctgggtgctg | 52 |
| | | ctttgtctcctgggagcaggctcagtggag | |
| | | actggagtcacccaaagtcccacacacctg | |
| | | atcaaaacgagaggacagcaagtgactctg | |
| | | agatgctcttctcagtctgggcacaacact | |
| | | gtgtcctggtaccaacaggccctgggtcag | |
| | | gggccccagtttatctttcagtattatagg | |
| | | gaggaagagaatggcagaggaaacttccct | |
| | | cctagattctcaggtctccagttccctaat | |
| | | tatagctctgagctgaatgtgaacgccttg | |
| | | gagctggacgactcggccctgtatctctgt | |
| | | gccagcagctgggaaagggcggggaaagct | |
| | | ttctttggacaaggcaccagactcacagtt | |
| | | gtagaggacctgaacaaggtgttcccaccc | |
| | | gaggtcgctgtgtttgagccatcagaagca | |
| | | gagatctcccacacccaaaaggccacactg | |
| | | gtgtgcctggccacaggcttcttccctgac | |
| | | cacgtggagctgagctggtgggtgaatggg | |
| | | aaggaggtgcacagtggggtcagcacggac | |
| | | ccgcagcccctcaaggagcagcccgccctc | |
| | | aatgactccagatactgcctgagcagccgc | |
| | | ctgagggtctcggccaccttctggcagaac | |
| | | ccccgcaaccacttccgctgtcaagtccag | |
| | | ttctacgggctctcggagaatgacgagtgg | |
| | | acccaggatagggccaaacccgtcacccag | |
| | | atcgtcagcgccgaggcctggggtagagca | |
| | | gactgtggctttacctcggtgtcctaccag | |
| | | caaggggtcctgtctgccaccatcctctat | |
| | | gagatcctgctagggaaggccaccctgtat | |
| | | gctgtgctggtcagcgcccttgtgttgatg | |
| | | gccatggtcaagagaaaggatttcTAA | |
| 66-4- | ALPHA: | Atgatgaaatccttgagagttttactggtg | 53 |
| KRAS-3 | | atcctgtggcttcagttaagctgggtttgg | |
| | | agccaacagaaggaggtggagcaggatcct | |
| | | ggaccactcagtgttccagagggagccatt | |
| | | gtttctctcaactgcacttacagcaacagt | |
| | | gcttttcaatacttcatgtggtacagacag | |
| | | tattccagaaaaggccctgagttgctgatg | |
| | | tacacatactccagtggtaacaaagaagat | |
| | | ggaaggtttacagcacaggtcgataaatcc | |
| | | agcaagtatatctccttgttcatcagagac | |
| | | tcacagcccagtgattcagccacctacctc | |
| | | tgtgcaatgagcgtctttatttatagcaca | |
| | | ttcatctttgggagtgggacaagattatca | |
| | | gtaaaacctgatatccagaaccctgaccct | |
| | | gccgtgtaccagctgagagactctaaatcc | |
| | | agtgacaagtctgtctgcctattcaccgat | |
| | | tttgattctcaaacaaatgtgtcacaaagt | |
| | | aaggattctgatgtgtatatcacagacaaa | |
| | | actgtgctagacatgaggtctatggacttc | |
| | | aagagcaacagtgctgtggcctggagcaac | |
| | | aaatctgactttgcatgtgcaaacgccttc | |
| | | aacaacagcattattccagaagacaccttc | |
| | | ttccccagcccagaaagttcctgtgatgtc | |
| | | aagctggtcgagaaaagctttgaaacagat | |
| | | acgaacctaaactttcaaaacctgtcagtg | |
| | | attgggttccgaatcctcctcctgaaagtg | |
| | | gccgggtttaatctgctcatgacgctgcgg | |
| | | ctgtggtccagcTAA | |
| | BETA: | Atgggaatcaggctcctgtgtcgtgtggcc | 54 |
| | | ttttgtttcctggctgtaggcctcgtagat | |
| | | gtgaaagtaacccagagctcgagatatcta | |
| | | gtcaaaaggacgggagagaaagtttttctg | |
| | | gaatgtgtccaggatatggaccatgaaaat | |
| | | atgttctggtatcgacaagacccaggtctg | |
| | | gggctacggctgatctatttctcatatgat | |
| | | gttaaaatgaaagaaaaaggagatattcct | |
| | | gaggggtacagtgtctctagagagaagaag | |
| | | gagcgcttctccctgattctggagtccgcc | |
| | | agcaccaaccagacatctatgtacctctgt | |
| | | gccagctctgggaggcaagagacccagtac | |
| | | ttcgggccaggcacgcggctcctggtgctc | |
| | | gaggacctgaacaaggtgttcccacccgag | |
| | | gtcgctgtgtttgagccatcagaagcagag | |
| | | atctcccacacccaaaaggccacactggtg | |
| | | tgcctggccacaggcttcttccctgaccac | |
| | | gtggagctgagctggtgggtgaatgggaag | |
| | | gaggtgcacagtggggtcagcacggacccg | |
| | | cagcccctcaaggagcagcccgccctcaat | |
| | | gactccagatactgcctgagcagccgcctg | |
| | | agggtctcggccaccttctggcagaacccc | |
| | | cgcaaccacttccgctgtcaagtccagttc | |
| | | tacgggctctcggagaatgacgagtggacc | |
| | | caggatagggccaaacccgtcacccagatc | |
| | | gtcagcgccgaggcctggggtagagcagac | |
| | | tgtggctttacctcggtgtcctaccagcaa | |
| | | ggggtcctgtctgccaccatcctctatgag | |
| | | atcctgctagggaaggccaccctgtatgct | |
| | | gtgctggtcagcgcccttgtgttgatggcc | |
| | | atggtcaagagaaaggatttcTAA | |
| 66-4- | ALPHA: | Atggaaactctcctgggagtgtctttggtg | 55 |
| KRAS-6 | | attctatggcttcaactggctagggtgaac | |
| | | agtcaacagggagaagaggatcctcaggcc | |
| | | ttgagcatccaggagggtgaaaatgccacc | |
| | | atgaactgcagttacaaaactagtataaac | |
| | | aatttacagtggtatagacaaaattcaggt | |
| | | agaggccttgtccacctaattttaatacgt | |
| | | tcaaatgaaagagagaaacacagtggaaga | |
| | | ttaagagtcacgcttgacacttccaagaaa | |
| | | agcagttccttgttgatcacggcttcccgg | |
| | | gcagcagacactgcttcttacttctgtgct | |
| | | acggacgcgtacacaaggcaactgaccttt | |
| | | ggatctgggacacaattgactgttttacct | |
| | | gatatccagaaccctgaccctgccgtgtac | |
| | | cagctgagagactctaaatccagtgacaag | |
| | | tctgtctgcctattcaccgattttgattct | |
| | | caaacaaatgtgtcacaaagtaaggattct | |
| | | gatgtgtatatcacagacaaaactgtgcta | |
| | | gacatgaggtctatggacttcaagagcaac | |
| | | agtgctgtggcctggagcaacaaatctgac | |
| | | tttgcatgtgcaaacgccttcaacaacagc | |
| | | attattccagaagacaccttcttccccagc | |
| | | ccagaaagttcctgtgatgtcaagctggtc | |
| | | gagaaaagctttgaaacagatacgaaccta | |
| | | aactttcaaaacctgtcagtgattgggttc | |
| | | cgaatcctcctcctgaaagtggccgggttt | |
| | | aatctgctcatgacgctgcggctgtggtcc | |
| | | agcTAA | |
| | BETA: | Atgagcaaccaggtgctctgctgtgtggtc | 56 |
| | | ctttgtctcctgggagcaaacaccgtggat | |
| | | ggtggaatcactcagtccccaaagtacctg | |
| | | ttcagaaaggaaggacagaatgtgaccctg | |
| | | agttgtgaacagaatttgaaccacgatgcc | |
| | | atgtactggtaccgacaggacccagggcaa | |
| | | gggctgagattgatctactactcacagata | |
| | | gtaaatgactttcagaaaggagatatagct | |
| | | gaagggtacagcgtctctcgggagaagaag | |
| | | gaatcctttcctctcactgtgacatcggcc | |
| | | caaaagaacccgacagctttctatctctgt | |
| | | gccagtgggggaccgggggctaacaggccc | |
| | | cagcattttggtgatgggactcgactctcc | |
| | | atcctagaggacctgaacaaggtgttccca | |
| | | cccgaggtcgctgtgtttgagccatcagaa | |
| | | gcagagatctcccacacccaaaaggccaca | |
| | | ctggtgtgcctggccacaggcttcttccct | |
| | | gaccacgtggagctgagctggtgggtgaat | |
| | | gggaaggaggtgcacagtggggtcagcacg | |
| | | gacccgcagcccctcaaggagcagcccgcc | |
| | | ctcaatgactccagatactgcctgagcagc | |
| | | cgcctgagggtctcggccaccttctggcag | |
| | | aacccccgcaaccacttccgctgtcaagtc | |
| | | cagttctacgggctctcggagaatgacgag | |
| | | tggacccaggatagggccaaacccgtcacc | |
| | | cagatcgtcagcgccgaggcctggggtaga | |
| | | gcagactgtggctttacctcggtgtcctac | |
| | | cagcaaggggtcctgtctgccaccatcctc | |
| | | tatgagatcctgctagggaaggccaccctg | |
| | | tatgctgtgctggtcagcgcccttgtgttg | |
| | | atggccatggtcaagagaaaggatttcTAA | |
| 66-4- | ALPHA: | Atggcatgccctggcttcctgtgggcactt | 57 |
| KRAS-8 | | gtgatctccacctgtcttgaatttagcatg | |
| | | gctcagacagtcactcagtctcaaccagag | |
| | | atgtctgtgcaggaggcagagaccgtgacc | |
| | | ctgagctgcacatatgacaccagtgagagt | |
| | | gattattatttattctggtacaagcagcct | |
| | | cccagcaggcagatgattctcgttattcgc | |
| | | caagaagcttataagcaacagaatgcaaca | |
| | | gagaatcgtttctctgtgaacttccagaaa | |
| | | gcagccaaatccttcagtctcaagatctca | |
| | | gactcacagctgggggatgccgcgatgtat | |
| | | ttctgtgcttataggagcctttgggggtcc | |
| | | gggtatgcactcaacttcggcaaaggcacc | |
| | | tcgctgttggtcacaccccatatccagaac | |
| | | cctgaccctgccgtgtaccagctgagagac | |
| | | tctaaatccagtgacaagtctgtctgccta | |
| | | ttcaccgattttgattctcaaacaaatgtg | |
| | | tcacaaagtaaggattctgatgtgtatatc | |
| | | acagacaaaactgtgctagacatgaggtct | |
| | | atggacttcaagagcaacagtgctgtggcc | |
| | | tggagcaacaaatctgactttgcatgtgca | |
| | | aacgccttcaacaacagcattattccagaa | |
| | | gacaccttcttccccagcccagaaagttcc | |
| | | tgtgatgtcaagctggtcgagaaaagcttt | |
| | | gaaacagatacgaacctaaactttcaaaac | |
| | | ctgtcagtgattgggttccgaatcctcctc | |
| | | ctgaaagtggccgggtttaatctgctcatg | |
| | | acgctgcggctgtggtccagcTAA | |
| | BETA: | Atgctgagtcttctgctccttctcctggga | 58 |
| | | ctaggctctgtgttcagtgctgtcatctct | |
| | | caaaagccaagcagggatatctgtcaacgt | |
| | | ggaacctccctgacgatccagtgtcaagtc | |
| | | gatagccaagtcaccatgatgttctggtac | |
| | | cgtcagcaacctggacagagcctgacactg | |
| | | atcgcaactgcaaatcagggctctgaggcc | |
| | | acatatgagagtggatttgtcattgacaag | |
| | | tttcccatcagccgcccaaacctaacattc | |
| | | tcaactctgactgtgagcaacatgagccct | |
| | | gaagacagcagcatatatctctgcagcgtt | |
| | | gaaaagggcgcgcaagagacccagtacttc | |
| | | gggccaggcacgcggctcctggtgctcgag | |
| | | gacctgaacaaggtgttcccacccgaggtc | |
| | | gctgtgtttgagccatcagaagcagagatc | |
| | | tcccacacccaaaaggccacactggtgtgc | |
| | | ctggccacaggcttcttccctgaccacgtg | |
| | | gagctgagctggtgggtgaatgggaaggag | |
| | | gtgcacagtggggtcagcacggacccgcag | |
| | | cccctcaaggagcagcccgccctcaatgac | |
| | | tccagatactgcctgagcagccgcctgagg | |
| | | gtctcggccaccttctggcagaacccccgc | |
| | | aaccacttccgctgtcaagtccagttctac | |
| | | gggctctcggagaatgacgagtggacccag | |
| | | gatagggccaaacccgtcacccagatcgtc | |
| | | agcgccgaggcctggggtagagcagactgt | |
| | | ggctttacctcggtgtcctaccagcaaggg | |
| | | gtcctgtctgccaccatcctctatgagatc | |
| | | ctgctagggaaggccaccctgtatgctgtg | |
| | | ctggtcagcgcccttgtgttgatggccatg | |
| | | gtcaagagaaaggatttcTAA | |
| 66-4- | ALPHA: | Atgacatccattcgagctgtatttatattc | 59 |
| KRAS-11 | | ctgtggctgcagctggacttggtgaatgga | |
| | | gagaatgtggagcagcatccttcaaccctg | |
| | | agtgtccaggagggagacagcgctgttatc | |
| | | aagtgtacttattcagacagtgcctcaaac | |
| | | tacttcccttggtataagcaagaacttgga | |
| | | aaaggacctcagcttattatagacattcgt | |
| | | tcaaatgtgggcgaaaagaaagaccaacga | |
| | | attgctgttacattgaacaagacagccaaa | |
| | | catttctccctgcacatcacagagacccaa | |
| | | cctgaagactcggctgtctacttctgtgca | |
| | | gcactctttggaaatgagaaattaaccttt | |
| | | gggactggaacaagactcaccatcataccc | |
| | | aatatccagaaccctgaccctgccgtgtac | |
| | | cagctgagagactctaaatccagtgacaag | |
| | | tctgtctgcctattcaccgattttgattct | |
| | | caaacaaatgtgtcacaaagtaaggattct | |
| | | gatgtgtatatcacagacaaaactgtgcta | |
| | | gacatgaggtctatggacttcaagagcaac | |
| | | agtgctgtggcctggagcaacaaatctgac | |
| | | tttgcatgtgcaaacgccttcaacaacagc | |
| | | attattccagaagacaccttcttccccagc | |
| | | ccagaaagttcctgtgatgtcaagctggtc | |
| | | gagaaaagctttgaaacagatacgaaccta | |
| | | aactttcaaaacctgtcagtgattgggttc | |
| | | cgaatcctcctcctgaaagtggccgggttt | |
| | | aatctgctcatgacgctgcggctgtggtcc | |
| | | agcTAA | |
| | BETA: | Atgggcaccaggctcctcttctgggtggcc | 60 |
| | | ttctgtctcctgggggcagatcacacagga | |
| | | gctggagtctcccagtcccccagtaacaag | |
| | | gtcacagagaagggaaaggatgtagagctc | |
| | | aggtgtgatccaatttcaggtcatactgcc | |
| | | ctttactggtaccgacagagcctggggcag | |
| | | ggcctggagtttttaatttacttccaaggc | |
| | | aacagtgcaccagacaaatcagggctgccc | |
| | | agtgatcgcttctctgcagagaggactggg | |
| | | ggatccgtctccactctgacgatccagcgc | |
| | | acacagcaggaggactcggccgtgtatctc | |
| | | tgtgccaccctacagggttggtcttataat | |
| | | gagcagttcttcgggccagggacacggctc | |
| | | accgtgctagaggacctgaacaaggtgttc | |
| | | ccacccgaggtcgctgtgtttgagccatca | |
| | | gaagcagagatctcccacacccaaaaggcc | |
| | | acactggtgtgcctggccacaggcttcttc | |
| | | cctgaccacgtggagctgagctggtgggtg | |
| | | aatgggaaggaggtgcacagtggggtcagc | |
| | | acggacccgcagcccctcaaggagcagccc | |
| | | gccctcaatgactccagatactgcctgagc | |
| | | agccgcctgagggtctcggccaccttctgg | |
| | | cagaacccccgcaaccacttccgctgtcaa | |
| | | gtccagttctacgggctctcggagaatgac | |
| | | gagtggacccaggatagggccaaacccgtc | |
| | | acccagatcgtcagcgccgaggcctggggt | |
| | | agagcagactgtggctttacctcggtgtcc | |
| | | taccagcaaggggtcctgtctgccaccatc | |
| | | ctctatgagatcctgctagggaaggccacc | |
| | | ctgtatgctgtgctggtcagcgcccttgtg | |
| | | ttgatggccatggtcaagagaaaggatttc | |
| | | TAA | |
| 66-4- | ALPHA: | Atgaagcccaccctcatctcagtgcttgtg | 61 |
| KRAS-12 | | ataatatttatactcagaggaacaagagcc | |
| | | cagagagtgactcagcccgagaagctcctc | |
| | | tctgtctttaaaggggccccagtggagctg | |
| | | aagtgcaactattcctattctgggagtcct | |
| | | gaactcttctggtatgtccagtactccaga | |
| | | caacgcctccagttactcttgagacacatc | |
| | | tctagagagagcatcaaaggcttcactgct | |
| | | gaccttaacaaaggcgagacatctttccac | |
| | | ctgaagaaaccatttgctcaagaggaagac | |
| | | tcagccatgtattactgtgctctaagtact | |
| | | ggaggcttcaaaactatctttggagcagga | |
| | | acaagactatttgttaaagcaaatatccag | |
| | | aaccctgaccctgccgtgtaccagctgaga | |
| | | gactctaaatccagtgacaagtctgtctgc | |
| | | ctattcaccgattttgattctcaaacaaat | |
| | | gtgtcacaaagtaaggattctgatgtgtat | |
| | | atcacagacaaaactgtgctagacatgagg | |
| | | tctatggacttcaagagcaacagtgctgtg | |
| | | gcctggagcaacaaatctgactttgcatgt | |
| | | gcaaacgccttcaacaacagcattattcca | |
| | | gaagacaccttcttccccagcccagaaagt | |
| | | tcctgtgatgtcaagctggtcgagaaaagc | |
| | | tttgaaacagatacgaacctaaactttcaa | |
| | | aacctgtcagtgattgggttccgaatcctc | |
| | | ctcctgaaagtggccgggtttaatctgctc | |
| | | atgacgctgcggctgtggtccagcTAA | |
| | BETA: | Atgctgagtcttctgctccttctcctggga | 62 |
| | | ctaggctctgtgttcagtgctgtcatctct | |
| | | caaaagccaagcagggatatctgtcaacgt | |
| | | ggaacctccctgacgatccagtgtcaagtc | |
| | | gatagccaagtcaccatgatgttctggtac | |
| | | cgtcagcaacctggacagagcctgacactg | |
| | | atcgcaactgcaaatcagggctctgaggcc | |
| | | acatatgagagtggatttgtcattgacaag | |
| | | tttcccatcagccgcccaaacctaacattc | |
| | | tcaactctgactgtgagcaacatgagccct | |
| | | gaagacagcagcatatatctctgcagcgtt | |
| | | ttgggtcccgggacagggggacgaggagct | |
| | | aactatggctacaccttcggttcggggacc | |
| | | aggttaaccgttgtagaggacctgaacaag | |
| | | gtgttcccacccgaggtcgctgtgtttgag | |
| | | ccatcagaagcagagatctcccacacccaa | |
| | | aaggccacactggtgtgcctggccacaggc | |
| | | ttcttccctgaccacgtggagctgagctgg | |
| | | tgggtgaatgggaaggaggtgcacagtggg | |
| | | gtcagcacggacccgcagcccctcaaggag | |
| | | cagcccgccctcaatgactccagatactgc | |
| | | ctgagcagccgcctgagggtctcggccacc | |
| | | ttctggcagaacccccgcaaccacttccgc | |
| | | tgtcaagtccagttctacgggctctcggag | |
| | | aatgacgagtggacccaggatagggccaaa | |
| | | cccgtcacccagatcgtcagcgccgaggcc | |
| | | tggggtagagcagactgtggctttacctcg | |
| | | gtgtcctaccagcaaggggtcctgtctgcc | |
| | | accatcctctatgagatcctgctagggaag | |
| | | gccaccctgtatgctgtgctggtcagcgcc | |
| | | cttgtgttgatggccatggtcaagagaaag | |
| | | gatttcTAA | |
| 66-4- | ALPHA: | Atggaaactctcctgggagtgtctttggtg | 63 |
| KRAS-13 | | attctatggcttcaactggctagggtgaac | |
| | | agtcaacagggagaagaggatcctcaggcc | |
| | | ttgagcatccaggagggtgaaaatgccacc | |
| | | atgaactgcagttacaaaactagtataaac | |
| | | aatttacagtggtatagacaaaattcaggt | |
| | | agaggccttgtccacctaattttaatacgt | |
| | | tcaaatgaaagagagaaacacagtggaaga | |
| | | ttaagagtcacgcttgacacttccaagaaa | |
| | | agcagttccttgttgatcacggcttcccgg | |
| | | gcagcagacactgcttcttacttctgtgct | |
| | | accactatagatggccagaagctgctcttt | |
| | | gcaaggggaaccatgttaaaggtggatctt | |
| | | aatatccagaaccctgaccctgccgtgtac | |
| | | cagctgagagactctaaatccagtgacaag | |
| | | tctgtctgcctattcaccgattttgattct | |
| | | caaacaaatgtgtcacaaagtaaggattct | |
| | | gatgtgtatatcacagacaaaactgtgcta | |
| | | gacatgaggtctatggacttcaagagcaac | |
| | | agtgctgtggcctggagcaacaaatctgac | |
| | | tttgcatgtgcaaacgccttcaacaacagc | |
| | | attattccagaagacaccttcttccccagc | |
| | | ccagaaagttcctgtgatgtcaagctggtc | |
| | | gagaaaagctttgaaacagatacgaaccta | |
| | | aactttcaaaacctgtcagtgattgggttc | |
| | | cgaatcctcctcctgaaagtggccgggttt | |
| | | aatctgctcatgacgctgcggctgtggtcc | |
| | | agcTAA | |
| | BETA: | Atgctgctgcttctgctgcttctggggcca | 64 |
| | | gcaggctccgggcttggtgctgtcgtctct | |
| | | caacatccgagcagggttatctgtaagagt | |
| | | ggaacctctgtgaagatcgagtgccgttcc | |
| | | ctggactttcaggccacaactatgttttgg | |
| | | tatcgtcagttcccgaaaaagagtctcatg | |
| | | ctgatggcaacttccaatgagggctccaag | |
| | | gccacatacgagcaaggcgtcgagaaggac | |
| | | aagtttctcatcaaccatgcaagcctgacc | |
| | | ttgtccactctgacagtgaccagtgcccat | |
| | | cctgaagacagcagcttctacatctgcagt | |
| | | gcttctgacagggggtccggggagctgttt | |
| | | tttggagaaggctctaggctgaccgtactg | |
| | | gaggacctgaacaaggtgttcccacccgag | |
| | | gtcgctgtgtttgagccatcagaagcagag | |
| | | atctcccacacccaaaaggccacactggtg | |
| | | tgcctggccacaggcttcttccctgaccac | |
| | | gtggagctgagctggtgggtgaatgggaag | |
| | | gaggtgcacagtggggtcagcacggacccg | |
| | | cagcccctcaaggagcagcccgccctcaat | |
| | | gactccagatactgcctgagcagccgcctg | |
| | | agggtctcggccaccttctggcagaacccc | |
| | | cgcaaccacttccgctgtcaagtccagttc | |
| | | tacgggctctcggagaatgacgagtggacc | |
| | | caggatagggccaaacccgtcacccagatc | |
| | | gtcagcgccgaggcctggggtagagcagac | |
| | | tgtggctttacctcggtgtcctaccagcaa | |
| | | ggggtcctgtctgccaccatcctctatgag | |
| | | atcctgctagggaaggccaccctgtatgct | |
| | | gtgctggtcagcgcccttgtgttgatggcc | |
| | | atggtcaagagaaaggatttcTAA | |
| 66-4- | ALPHA: | Atggaaactctcctgggagtgtctttggtg | 65 |
| KRAS-14 | | attctatggcttcaactggctagggtgaac | |
| | | agtcaacagggagaagaggatcctcaggcc | |
| | | ttgagcatccaggagggtgaaaatgccacc | |
| | | atgaactgcagttacaaaactagtataaac | |
| | | aatttacagtggtatagacaaaattcaggt | |
| | | agaggccttgtccacctaattttaatacgt | |
| | | tcaaatgaaagagagaaacacagtggaaga | |
| | | ttaagagtcacgcttgacacttccaagaaa | |
| | | agcagttccttgttgatcacggcttcccgg | |
| | | gcagcagacactgcttcttacttctgtgct | |
| | | acggacgcatacaatgccagactcatgttt | |
| | | ggagatggaactcagctggtggtgaagccc | |
| | | aatatccagaaccctgaccctgccgtgtac | |
| | | cagctgagagactctaaatccagtgacaag | |
| | | tctgtctgcctattcaccgattttgattct | |
| | | caaacaaatgtgtcacaaagtaaggattct | |
| | | gatgtgtatatcacagacaaaactgtgcta | |
| | | gacatgaggtctatggacttcaagagcaac | |
| | | agtgctgtggcctggagcaacaaatctgac | |
| | | tttgcatgtgcaaacgccttcaacaacagc | |
| | | attattccagaagacaccttcttccccagc | |
| | | ccagaaagttcctgtgatgtcaagctggtc | |
| | | gagaaaagctttgaaacagatacgaaccta | |
| | | aactttcaaaacctgtcagtgattgggttc | |
| | | cgaatcctcctcctgaaagtggccgggttt | |
| | | aatctgctcatgacgctgcggctgtggtcc | |
| | | agcTAA | |
| | BETA: | Atgctgagtcttctgctccttctcctggga | 66 |
| | | ctaggctctgtgttcagtgctgtcatctct | |
| | | caaaagccaagcagggatatctgtcaacgt | |
| | | ggaacctccctgacgatccagtgtcaagtc | |
| | | gatagccaagtcaccatgatgttctggtac | |
| | | cgtcagcaacctggacagagcctgacactg | |
| | | atcgcaactgcaaatcagggctctgaggcc | |
| | | acatatgagagtggatttgtcattgacaag | |
| | | tttcccatcagccgcccaaacctaacattc | |
| | | tcaactctgactgtgagcaacatgagccct | |
| | | gaagacagcagcatatatctctgcagcgtt | |
| | | gtggcaagcgggagtgtagatacgcagtat | |
| | | tttggcccaggcacccggctgacagtgctc | |
| | | gaggacctgaacaaggtgttcccacccgag | |
| | | gtcgctgtgtttgagccatcagaagcagag | |
| | | atctcccacacccaaaaggccacactggtg | |
| | | tgcctggccacaggcttcttccctgaccac | |
| | | gtggagctgagctggtgggtgaatgggaag | |
| | | gaggtgcacagtggggtcagcacggacccg | |
| | | cagcccctcaaggagcagcccgccctcaat | |
| | | gactccagatactgcctgagcagccgcctg | |
| | | agggtctcggccaccttctggcagaacccc | |
| | | cgcaaccacttccgctgtcaagtccagttc | |
| | | tacgggctctcggagaatgacgagtggacc | |
| | | caggatagggccaaacccgtcacccagatc | |
| | | gtcagcgccgaggcctggggtagagcagac | |
| | | tgtggctttacctcggtgtcctaccagcaa | |
| | | ggggtcctgtctgccaccatcctctatgag | |
| | | atcctgctagggaaggccaccctgtatgct | |
| | | gtgctggtcagcgcccttgtgttgatggcc | |
| | | atggtcaagagaaaggatttcTAA | |
| 66-4- | ALPHA: | Atggtcctgaaattctccgtgtccattctt | 67 |
| KRAS-19 | | tggattcagttggcatgggtgagcacccag | |
| | | ctgctggagcagagccctcagtttctaagc | |
| | | atccaagagggagaaaatctcactgtgtac | |
| | | tgcaactcctcaagtgttttttccagctta | |
| | | caatggtacagacaggagcctggggaaggt | |
| | | cctgtcctcctggtgacagtagttacgggt | |
| | | ggagaagtgaagaagctgaagagactaacc | |
| | | tttcagtttggtgatgcaagaaaggacagt | |
| | | tctctccacatcactgcagcccagcctggt | |
| | | gatacaggcctctacctctgtgcaggtcct | |
| | | gcgggagcccagaagctggtatttggccaa | |
| | | ggaaccaggctgactatcaacccaaatatc | |
| | | cagaaccctgaccctgccgtgtaccagctg | |
| | | agagactctaaatccagtgacaagtctgtc | |
| | | tgcctattcaccgattttgattctcaaaca | |
| | | aatgtgtcacaaagtaaggattctgatgtg | |
| | | tatatcacagacaaaactgtgctagacatg | |
| | | aggtctatggacttcaagagcaacagtgct | |
| | | gtggcctggagcaacaaatctgactttgca | |
| | | tgtgcaaacgccttcaacaacagcattatt | |
| | | ccagaagacaccttcttccccagcccagaa | |
| | | agttcctgtgatgtcaagctggtcgagaaa | |
| | | agctttgaaacagatacgaacctaaacttt | |
| | | caaaacctgtcagtgattgggttccgaatc | |
| | | ctcctcctgaaagtggccgggtttaatctg | |
| | | ctcatgacgctgcggctgtggtccagcTAA | |
| | BETA: | Atgagcatcagcctcctgtgctgtgcagcc | 68 |
| | | tttcctctcctgtgggcaggtccagtgaat | |
| | | gctggtgtcactcagaccccaaaattccgc | |
| | | atcctgaagataggacagagcatgacactg | |
| | | cagtgtacccaggatatgaaccataactac | |
| | | atgtactggtatcgacaagacccaggcatg | |
| | | gggctgaagctgatttattattcagttggt | |
| | | gctggtatcactgataaaggagaagtcccg | |
| | | aatggctacaacgtctccagatcaaccaca | |
| | | gaggatttcccgctcaggctggagttggct | |
| | | gctccctcccagacatctgtgtacttctgt | |
| | | gccagcagccccgtaccctactctggaaac | |
| | | accatatattttggagagggaagttggctc | |
| | | actgttgtagaggacctgaacaaggtgttc | |
| | | ccacccgaggtcgctgtgtttgagccatca | |
| | | gaagcagagatctcccacacccaaaaggcc | |
| | | acactggtgtgcctggccacaggcttcttc | |
| | | cctgaccacgtggagctgagctggtgggtg | |
| | | aatgggaaggaggtgcacagtggggtcagc | |
| | | acggacccgcagcccctcaaggagcagccc | |
| | | gccctcaatgactccagatactgcctgagc | |
| | | agccgcctgagggtctcggccaccttctgg | |
| | | cagaacccccgcaaccacttccgctgtcaa | |
| | | gtccagttctacgggctctcggagaatgac | |
| | | gagtggacccaggatagggccaaacccgtc | |
| | | acccagatcgtcagcgccgaggcctggggt | |
| | | agagcagactgtggctttacctcggtgtcc | |
| | | taccagcaaggggtcctgtctgccaccatc | |
| | | ctctatgagatcctgctagggaaggccacc | |
| | | ctgtatgctgtgctggtcagcgcccttgtg | |
| | | ttgatggccatggtcaagagaaaggatttc | |
| | | TAA | |
| 66-4- | ALPHA: | Atgctcctgctgctcgtcccagtgctcgag | 69 |
| KRAS-22 | | gtgatttttaccctgggaggaaccagagcc | |
| | | cagtcggtgacccagcttggcagccacgtc | |
| | | tctgtctctgaaggagccctggttctgctg | |
| | | aggtgcaactactcatcgtctgttccacca | |
| | | tatctcttctggtatgtgcaataccccaac | |
| | | caaggactccagcttctcctgaagtacaca | |
| | | tcagcggccaccctggttaaaggcatcaac | |
| | | ggttttgaggctgaatttaagaagagtgaa | |
| | | acctccttccacctgacgaaaccctcagcc | |
| | | catatgagcgacgcggctgagtacttctgt | |
| | | gctgtgagtaagagtgacaagatcatcttt | |
| | | ggaaaagggacacgacttcatattctcccc | |
| | | aatatccagaaccctgaccctgccgtgtac | |
| | | cagctgagagactctaaatccagtgacaag | |
| | | tctgtctgcctattcaccgattttgattct | |
| | | caaacaaatgtgtcacaaagtaaggattct | |
| | | gatgtgtatatcacagacaaaactgtgcta | |
| | | gacatgaggtctatggacttcaagagcaac | |
| | | agtgctgtggcctggagcaacaaatctgac | |
| | | tttgcatgtgcaaacgccttcaacaacagc | |
| | | attattccagaagacaccttcttccccagc | |
| | | ccagaaagttcctgtgatgtcaagctggtc | |
| | | gagaaaagctttgaaacagatacgaaccta | |
| | | aactttcaaaacctgtcagtgattgggttc | |
| | | cgaatcctcctcctgaaagtggccgggttt | |
| | | aatctgctcatgacgctgcggctgtggtcc | |
| | | agcTAA | |
| | BETA: | Atggcctccctgctcttcttctgtggggcc | 70 |
| | | ttttatctcctgggaacagggtccatggat | |
| | | gctgatgttacccagaccccaaggaatagg | |
| | | atcacaaagacaggaaagaggattatgctg | |
| | | gaatgttctcagactaagggtcatgataga | |
| | | atgtactggtatcgacaagacccaggactg | |
| | | ggcctacggttgatctattactcctttgat | |
| | | gtcaaagatataaacaaaggagagatctct | |
| | | gatggatacagtgtctctcgacaggcacag | |
| | | gctaaattctccctgtccctagagtctgcc | |
| | | atccccaaccagacagctctttacttctgt | |
| | | gccaccagtgagggggggagtacgggcact | |
| | | gaagctttctttggacaaggcaccagactc | |
| | | acagttgtagaggacctgaacaaggtgttc | |
| | | ccacccgaggtcgctgtgtttgagccatca | |
| | | gaagcagagatctcccacacccaaaaggcc | |
| | | acactggtgtgcctggccacaggcttcttc | |
| | | cctgaccacgtggagctgagctggtgggtg | |
| | | aatgggaaggaggtgcacagtggggtcagc | |
| | | acggacccgcagcccctcaaggagcagccc | |
| | | gccctcaatgactccagatactgcctgagc | |
| | | agccgcctgagggtctcggccaccttctgg | |
| | | cagaacccccgcaaccacttccgctgtcaa | |
| | | gtccagttctacgggctctcggagaatgac | |
| | | gagtggacccaggatagggccaaacccgtc | |
| | | acccagatcgtcagcgccgaggcctggggt | |
| | | agagcagactgtggctttacctcggtgtcc | |
| | | taccagcaaggggtcctgtctgccaccatc | |
| | | ctctatgagatcctgctagggaaggccacc | |
| | | ctgtatgctgtgctggtcagcgcccttgtg | |
| | | ttgatggccatggtcaagagaaaggatttc | |
| | | TAA | |
| 66-4- | ALPHA: | Atgaactattctccaggcttagtatctctg | 71 |
| KRAS-25 | | atactcttactgcttggaagaacccgtgga | |
| | | aattcagtgacccagatggaagggccagtg | |
| | | actctctcagaagaggccttcctgactata | |
| | | aactgcacgtacacagccacaggataccct | |
| | | tcccttttctggtatgtccaatatcctgga | |
| | | gaaggtctacagctcctcctgaaagccacg | |
| | | aaggctgatgacaagggaagcaacaaaggt | |
| | | tttgaagccacataccgtaaagaaaccact | |
| | | tctttccacttggagaaaggctcagttcaa | |
| | | gtgtcagactcagcggtgtacttctgtgct | |
| | | ctgaataggaacaccggtaaccagttctat | |
| | | tttgggacagggacaagtttgacggtcatt | |
| | | ccaaatatccagaaccctgaccctgccgtg | |
| | | taccagctgagagactctaaatccagtgac | |
| | | aagtctgtctgcctattcaccgattttgat | |
| | | tctcaaacaaatgtgtcacaaagtaaggat | |
| | | tctgatgtgtatatcacagacaaaactgtg | |
| | | ctagacatgaggtctatggacttcaagagc | |
| | | aacagtgctgtggcctggagcaacaaatct | |
| | | gactttgcatgtgcaaacgccttcaacaac | |
| | | agcattattccagaagacaccttcttcccc | |
| | | agcccagaaagttcctgtgatgtcaagctg | |
| | | gtcgagaaaagctttgaaacagatacgaac | |
| | | ctaaactttcaaaacctgtcagtgattggg | |
| | | ttccgaatcctcctcctgaaagtggccggg | |
| | | tttaatctgctcatgacgctgcggctgtgg | |
| | | tccagcTAA | |
| | BETA: | atgagcaaccaggtgctctgctgtgtggtc | 72 |
| | | ctttgtctcctgggagcaaacaccgtggat | |
| | | ggtggaatcactcagtccccaaagtacctg | |
| | | ttcagaaaggaaggacagaatgtgaccctg | |
| | | agttgtgaacagaatttgaaccacgatgcc | |
| | | atgtactggtaccgacaggacccagggcaa | |
| | | gggctgagattgatctactactcacagata | |
| | | gtaaatgactttcagaaaggagatatagct | |
| | | gaagggtacagcgtctctcgggagaagaag | |
| | | gaatcctttcctctcactgtgacatcggcc | |
| | | caaaagaacccgacagctttctatctctgt | |
| | | gccagtagtatcccacagggcaatggctac | |
| | | accttcggttcggggaccaggttaaccgtt | |
| | | gtagaggacctgaacaaggtgttcccaccc | |
| | | gaggtcgctgtgtttgagccatcagaagca | |
| | | gagatctcccacacccaaaaggccacactg | |
| | | gtgtgcctggccacaggcttcttccctgac | |
| | | cacgtggagctgagctggtgggtgaatggg | |
| | | aaggaggtgcacagtggggtcagcacggac | |
| | | ccgcagcccctcaaggagcagcccgccctc | |
| | | aatgactccagatactgcctgagcagccgc | |
| | | ctgagggtctcggccaccttctggcagaac | |
| | | ccccgcaaccacttccgctgtcaagtccag | |
| | | ttctacgggctctcggagaatgacgagtgg | |
| | | acccaggatagggccaaacccgtcacccag | |
| | | atcgtcagcgccgaggcctggggtagagca | |
| | | gactgtggctttacctcggtgtcctaccag | |
| | | caaggggtcctgtctgccaccatcctctat | |
| | | gagatcctgctagggaaggccaccctgtat | |
| | | gctgtgctggtcagcgcccttgtgttgatg | |
| | | gccatggtcaagagaaaggatttcTAA | |

TABLE 5
Nucleic acid sequences, TCR alpha VDJ and
CDR3 sequences and TCR beta VDJ and
CDR3 sequences.

| | | NT VDJ gene | | | |
|---|---|---|---|---|---|
| | | and CDR | | | |
| | | sequence | | NT CDR3 | |
| 66-4- | ALPHA: | TRAV8-4_GAGTGTA | 73 | tgtgctgtgagtgta | 75 |
| KRAS- | | GGTCCTGGC_TRAJ3 | | ggtcctggcaacaca | |
| 1 | | 7 | | ggcaaactaatcttt | |
| | BETA: | TRBV28_AGTTTGGG | 74 | tgtgccagcagtttg | 76 |
| | | GGTTCTAGGCCTGCG | | ggggttctaggcctg | |
| | | GTACT_TRBJ2-7 | | cggtacttc | |
| 66-4- | ALPHA: | TRAV13-2_TGCAG_ | 77 | tgtgcagagaatagt | 78 |
| KRAS- | | (AGAATA)_GTGGA_ | | ggaggtagcaactat | |
| 2 | | TRAJ53 | | aaactgacattt | |
| | BETA: | TRBV5-4_CTGGGAA | 79 | tgtgccagcagctgg | 80 |
| | | AGGGCGGGGAAAGCT | | gaaagggcggggaaa | |
| | | _TRBJ1-1 | | gctttcttt | |
| 66-4- | ALPHA: | TRAV12-3_GAGCGT | 81 | tgtgcaatgagcgtc | 82 |
| KRAS- | | CTTTATTTA_TRAJ1 | | tttatttatagcaca | |
| 3 | | 4 | | ttcatcttt | |
| | BETA: | TRBV28_CCAGCTCT | 83 | tgtgccagctctggg | 84 |
| | | GGGAGGCAAGA_TRB | | aggcaagagacccag | |
| | | J2-5 | | tacttc | |
| 66-4- | ALPHA: | TRAV17_GGACGCGT | 85 | tgtgctacggacgcg | 86 |
| KRAS- | | ACACAAGG_TRAJ22 | | tacacaaggcaactg | |
| 6 | | | | accttt | |
| | BETA: | TRBV19_CCAGTGGG | 87 | tgtgccagtggggga | 88 |
| | | GGACCGGGGGCTAAC | | ccgggggctaacagg | |
| | | AGGCCCC_TRBJ1-5 | | ccccagcatttt | |
| 66-4- | ALPHA: | TRAV38-2_DV8_GG | 89 | tgtgcttataggagc | 90 |
| KRAS- | | AGCCTTTGGGGGTCC | | ctttgggggtccggg | |
| 8 | | GG_TRAJ41 | | tatgcactcaacttc | |
| | BETA: | TRBV29-1_TTGAAA | 91 | tgcagcgttgaaaag | 92 |
| | | AGGGCGCGCAAGA_T | | ggcgcgcaagagacc | |
| | | RBJ2-5 | | cagtacttc | |
| 66-4- | ALPHA: | TRAV13-1_CAGCAC | 93 | tgtgcagcactcttt | 94 |
| KRAS- | | TCTTTG_TRAJ48 | | ggaaatgagaaatta | |
| 11 | | | | accttt | |
| | BETA: | TRBV7-2_TGCCACC | 95 | tgtgccaccctacag | 96 |
| | | CTACAGGGTTGGTCT | | ggttggtcttataat | |
| | | TATAATGA_TRBJ2- | | gagcagttcttc | |
| | | 1 | | | |
| 66-4- | ALPHA: | TRAV16_CTAAG_ | 97 | tgtgctctaagtact | 998 |
| KRAS- | | (T)_ACTGG_TRAJ9 | | ggaggcttcaaaact | |
| 12 | | | | atcttt | |
| | BETA: | TRBV29-1_GCGTTT | 99 | tgcagcgttttgggt | 100 |
| | | TGGGTCCCGGGACAG | | cccgggacaggggga | |
| | | GGGGACGAGGAGCTA | | cgaggagctaactat | |
| | | AC_TRBJ1-2 | | ggctacaccttc | |
| 66-4- | ALPHA: | TRAV17_GCTACCAC | 101 | tgtgctaccactata | 102 |
| KRAS- | | TATAGATG_TRAJ16 | | gatggccagaagctg | |
| 13 | | | | ctcttt | |
| | BETA: | TRBV20-1_GTGCTT | 103 | tgcagtgcttctgac | 104 |
| | | CTGACAGGGGGTCCG | | agggggtccggggag | |
| | | GG_TRBJ2-2 | | ctgtttttt | |
| 66-4- | ALPHA: | TRAV17_GGACGCAT | 105 | tgtgctacggacgca | 106 |
| KRAS- | | ACAAT_TRAJ31 | | tacaatgccagactc | |
| 14 | | | | atgttt | |
| | BETA: | TRBV29-1_CGTTGT | 107 | tgcagcgttgtggca | 108 |
| | | GGCAAGCGGGAGTGT | | agcgggagtgtagat | |
| | | AGATA_TRBJ2-3 | | acgcagtatttt | |
| 66-4- | ALPHA: | TRAV27_GCAGGTCC | 109 | tgtgcaggtcctgcg | 110 |
| KRAS- | | TGCGGGAG_TRAJ54 | | ggagcccagaagctg | |
| 19 | | | | gtattt | |
| | BETA: | TRBV6-6_AGCAGCC | 111 | tgtgccagcagcccc | 112 |
| | | CCGTACCCTACTCTG | | gtaccctactctgga | |
| | | _TRBJ1-_3 | | aacaccatatatttt | |
| 66-4- | ALPHA: | TRAV8-4_TGAGTAA | 113 | tgtgctgtgagtaag | 114 |
| KRAS- | | GAGTGACA_TRAJ30 | | agtgacaagatcatc | |
| 22 | | | | ttt | |
| | BETA: | TRBV24-1_AGTGAG | 115 | tgtgccaccagtgag | 116 |
| | | GGGGGGAGTACGGGC | | ggggggagtacgggc | |
| | | ACTG_TRBJ1-1 | | actgaagctttcttt | |
| 66-4- | ALPHA: | TRAV9-2_TCTGAAT | 117 | tgtgctctgaatagg | 118 |
| KRAS- | | AGGAACA_TRAJ49 | | aacaccggtaaccag | |
| 25 | | | | ttctatttt | |
| | BETA: | TRBV19_AGTATCCC | 119 | tgtgccagtagtatc | 120 |
| | | ACAGGGCAATGGC_ | | ccacagggcaatggc | |
| | | TRBJ1-2 | | tacaccttc | |

### Example 2. Cross-Presentation of Cell-Associated KRAS Protein

The purpose of this example is to express wildtype versus KRAS G12>V within cells, which are then used as the source of cell-associated antigen for cross-presenting CD1-phenotype dendritic cells (or other suitable antigen-presenting cell (APC) population) bearing the relevant HLA-DR allele in a form detectable by CD4+ T cells expressing TCR2.

The inventors have shown that TCR2 recognizes APC expressing the relevant HLA-DR allele when pulsed with the minimal 15 amino acid-long G12>V peptide (but not the wildtype), or when expressing in-vitro-transcribed RNA encoding a 50 amino-acid fragment of the KRAS G12>V (but not the wildtype) protein in a form that is preceded by a signal-sequence and followed by a membrane-targeting sequence. These observations confirm that TCR2 recognizes a peptide epitope that can be generated from a larger precursor protein. Thus, it is possible to determine if TCR2 recognizes the complete KRAS protein when cross-presented from a cellular source.

Study Design. (1) Generate antigen-expressing cells. Transfect 293T cells with KRAS G12>V-encoding MSGV1 retrovirus containing an eGFP transfection marker. Sort on eGFP+ cells and confirm KRAS expression by RNAseq. These cells can then be used as the source of cell-associated antigen for cross-presentation. (2) Generate cross-presenting APC. Follow published protocols to generate CD11c+monocyte-derived ‘DC1’ dendritic cells from appropriate donors expressing relevant HLA-DR alleles. (3) Co-culture KRAS-expressing 293T cells with DCL. 293T cells expressing KRAS G12>V may be prepared for use as cellular sources of antigen by freeze/thaw, irradiation, or centrifugation prior to co-culture with activated DC1 and allowed to acquire, process, and present the cell-associated antigen via HLA class II molecules. (4) Recognition assays with TCR2. TCR2 may be expressed in primary human CD4+ T cells using retrovirus transduction. T cells expressing this TCR may be isolated by positive selection and co-cultured with the DC1 “fed” with cell-associated KRAS G12>V (versus wildtype) protein. 6-24h later, recognition will be assessed by activation marker upregulation and/or cytokine production. If cross-presentation occurs, recognition of DC1 by TCR2 leads to the upregulation of activation markers such as 4-1BB or CD69 and by cytokine production such as IFN-γ.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. An engineered T cell receptor (TCR) comprising an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24, wherein the TCR is specific for a KRAS G12>V mutation peptide.

2. The TCR of claim 1, wherein the engineered TCR binds to the KRAS G12>V mutation peptide in a complex with HLA DRB5*01:01.

3. The TCR of claim 1, wherein the TCR comprises an alpha chain having at least 90, 95, 98, or 99% identity to the amino acid sequence of SEQ ID NO: 25, 27, 29, 31, 33, 35, 37, 39, 41, 43 and/or a beta chain having at least 90% identity to the amino acid sequence of SEQ ID NO: 26, 28, 30, 32, 34, 36, 38, 40, 42, and 44.

4. The TCR of claim 1, wherein the TCR is humanized.

5. The TCR of claim 1, wherein the TCR comprises an alpha chain having at least 90, 95, 98, or 99% identity to the nucleotide sequence of SEQ ID NO: 49, 51, 53, 55, 57, 59, 61, 63, 65, 67, 69, 71 and/or a beta chain having at least 95% identity to the nucleotide sequence of SEQ ID NO: 50, 52, 54, 56, 58, 60, 62, 64, 66, 68, 70, 72, and 74.

6. The TCR of claim 1, wherein the TCR is further defined as a soluble TCR, wherein the soluble TCR does not comprise a transmembrane domain, or comprises transmembrane domain that is a CD28 transmembrane domain or a CD8a transmembrane domain, or further comprises a T-cell signaling domain of any one of the following proteins: a human CD8-alpha protein, a human CD28 protein, a human CD3-zeta protein, a human FcRγ protein, a CD27 protein, an OX40 protein, a human 4-1BB protein, or any combination of the foregoing.

7. The TCR of any one of claims 1-6, the TCR further comprising a detectable label.

8. The TCR of any one of claims 1-6, wherein the TCR is covalently bound to a therapeutic agent, an immunotoxin or a chemotherapeutic agent.

9. The TCR of any one of claims 1-6, wherein the TCR does not recognize wild-type RAS, and the CDR3 is selected from SEQ ID NO: 1, 3, 5 and a beta chain CDR3 having the amino acid sequence of SEQ ID NO: 2, 4, 6.

10. The TCR of any one of claims 1-6, wherein the TCR is part of a multivalent TCR complex comprising a plurality of TCRs according to claim 1.

11. The complex of claim 10, wherein the multivalent TCR comprises 2, 3, 4 or more TCRs associated with one another; wherein the multivalent TCR is present in a lipid bilayer, in a liposome, or is attached to a nanoparticle; or wherein the TCRs are associated with one another via a linker molecule.

12. A polypeptide encoding the TCR of claims 1-6.

13. A polynucleotide encoding the polypeptide of any one of claims 1-6.

14. An expression vector encoding the TCR of any one of claims 1-6.

15. The expression vector of claim 14, wherein the sequence encoding the TCR is under the control of a promoter.

16. The expression vector of claim 14, wherein the expression vector is a viral or a retroviral vector.

17. The expression vector of claim 14, wherein the vector further encodes a linker domain positioned between the alpha chain and beta chain.

18. The expression vector of claim 17, wherein the linker domain comprises one or more protease cleavage sites, or wherein the one or more cleavage sites are separated by a spacer.

19. A host cell engineered to express the TCR of any one of claims 1-8.

20. The host cell of claim 19, wherein the cell is a T cell, NK cell, invariant NK cell, NKT cell, mesenchymal stem cell (MSC), or induced pluripotent stem (iPS) cell.

21. The host cell of claim 19, wherein the host cell is an immune cell.

22. The host cell of claim 19, wherein the T cell is a CD8⁺ T cell, CD4⁺ T cell, or γδ T cell.

23. The host cell of claim 19, wherein the T cell is a regulatory T cell (Treg).

24. The host cell of claim 19, wherein the host cell is autologous or allogeneic.

25. A method for engineering a host cell comprising contacting an immune cell with the TCR of any one of claims 1-8 or the expression vector of any one of claims 15-19.

26. The method of claim 27, wherein contacting is further defined as transfecting or transducing, wherein transfecting comprises electroporating RNA encoding the TCR of any one of claims 1-8 into the immune cell.

27. A method for treating a subject with a cancer comprising a KRAS G12>V mutation peptide, the method comprising:
administering to the subject an effective amount of one or more immune cells modified by cloning genes of the alpha and beta chains of a T cell receptor (TCR) ex vivo to express a chimeric antigen receptor specific for the KRAS G12>V mutation, wherein the chimeric antigen receptor comprises an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24.

28. The method of claim 27, wherein the immune cell is T cell, NK cell, invariant NK cell, NKT cell, mesenchymal stem cell (MSC), or induced pluripotent stem (iPS) cell, or a peripheral blood lymphocyte.

29. The method of claim 27, further comprising at least one of:
sorting the immune cells into T cells to isolate TCR engineered T cells;
performing a T cell cloning of the immune cells by serial dilution; or
expanding a T cell clone from the immune cells by a rapid expansion protocol.

30. The method of claim 27, wherein the subject is identified to have an HLA DRB5*01:01 allele.

31. The method of claim 27, wherein the immune cell is a T cell selected from a CD8⁺ T cell, CD4⁺ T cell, or Treg.

32. The method of claim 27, wherein the cancer is selected from colorectal cancer, pancreatic cancer, renal cancer, lung cancer, liver cancer, breast cancer, prostate cancer, gastrointestinal cancer, peritoneal cancer, melanoma, endometrial cancer, ovarian cancer, cervical cancer, uterine carcinoma, bladder cancer, glioblastoma, brain metastases, salivary gland carcinoma, thyroid cancer, brain cancer, lymphoma, myeloma, and head and neck cancer.

33. The method of claim 27, wherein the cancer is selected from pancreatic ductal adenocarcinoma and colorectal adenocarcinoma.

34. The method of claim 27, wherein the TCR engineered cells are autologous or allogeneic.

35. The method of claim 27, further comprising administering a second anticancer selected from chemotherapy, immunotherapy, surgery, radiotherapy, or biotherapy.

36. The method of claim 27, wherein the one or more immune cells are administered intravenously, intraperitoneally, intratracheally, intratumorally, intramuscularly, endoscopically, intralesionally, percutaneously, subcutaneously, regionally, or by direct injection or perfusion.

37. A chimeric antigen receptor expressing T cell (CAR-T) comprising an antigen recognition moiety and a T-cell activation moiety, wherein the T-cell activation moiety comprises a transmembrane domain, and wherein the antigen recognition moiety is directed against a KRAS G12>V mutation.

38. The CAR-T of claim 37, wherein the antigen recognition moiety does not recognize non-mutated RAS.

39. The CAR-T of claim 37, wherein the transmembrane domain is a CD28 transmembrane domain or a CD8a transmembrane domain.

40. The CAR-T of claim 37, wherein the T-cell activation moiety comprises a T-cell signaling domain of any one of the following proteins: a human CD8-alpha protein, a human CD28 protein, a human CD3-zeta protein, a human FcRγ protein, a CD27 protein, an OX40 protein, a human 4-1BB protein, or any combination of the foregoing.

41. The CAR-T of claim 37, wherein the antigen recognition moiety comprises the amino acid sequence of wherein the TCR comprises an alpha chain variable region having at least 90, 95, 98, or 99% identity to the amino acid sequence of SEQ ID NO: 25, 27, 29, 31, 33, 35, 37, 39, 41, 43 and/or a beta chain variable region having at least 90% identity to the amino acid sequence of SEQ ID NO: 26, 28, 30, 32, 34, 36, 38, 40, 42, and 44.

42. The CAR-T of claim 37, wherein the antigen recognition moiety comprises an alpha chain CDR3 having the amino acid sequence of SEQ ID NO: 1, 3, 5, 7, 9, 11, 13, 15, 17, 19, 21, or 23 and/or a beta chain CDR3 having the amino acid sequence of SEQ ID NO: 2, 4, 6, 8, 10, 12, 14, 16, 18, 20, 22, or 24.


SEQUENCE LISTING (120 sequences)

SEQ ID NO: 1 - protein, 15 aa, Artificial Sequence
  Synthetic
CAVSVGPGNTGKLIF

SEQ ID NO: 2 - protein, 13 aa, Artificial Sequence
  Synthetic
CASSLGVLGLRYF

SEQ ID NO: 3 - protein, 14 aa, Artificial Sequence
  Synthetic
CAENSGGSNYKLTF

SEQ ID NO: 4 - protein, 13 aa, Artificial Sequence
  Synthetic
CASSWERAGKAFF

SEQ ID NO: 5 - protein, 13 aa, Artificial Sequence
  Synthetic
CAMSVFIYSTFIF

SEQ ID NO: 6 - protein, 12 aa, Artificial Sequence
  Synthetic
CASSGRQETQYF

SEQ ID NO: 7 - protein, 12 aa, Artificial Sequence
  Synthetic
CATDAYTRQLTF

SEQ ID NO: 8 - protein, 14 aa, Artificial Sequence
  Synthetic
CASGGPGANRPQHF

SEQ ID NO: 9 - protein, 15 aa, Artificial Sequence
  Synthetic
CAYRSLWGSGYALNF

SEQ ID NO: 10 - protein, 13 aa, Artificial Sequence
  Synthetic
CSVEKGAQETQYF

SEQ ID NO: 11 - protein, 12 aa, Artificial Sequence
  Synthetic
CAALFGNEKLTF

SEQ ID NO: 12 - protein, 14 aa, Artificial Sequence
  Synthetic
CATLQGWSYNEQFF

SEQ ID NO: 13 - protein, 12 aa, Artificial Sequence
  Synthetic
CALSTGGFKTIF

SEQ ID NO: 14 - protein, 19 aa, Artificial Sequence
  Synthetic
CSVLGPGTGGRGANYGYTF

SEQ ID NO: 15 - protein, 12 aa, Artificial Sequence
  Synthetic
CATTIDGQKLLF

SEQ ID NO: 16 - protein, 13 aa, Artificial Sequence
  Synthetic
CSASDRGSGELFF

SEQ ID NO: 17 - protein, 12 aa, Artificial Sequence
  Synthetic
CATDAYNARLMF

SEQ ID NO: 18 - protein, 14 aa, Artificial Sequence
  Synthetic
CSVVASGSVDTQYF

SEQ ID NO: 19 - protein, 12 aa, Artificial Sequence
  Synthetic
CAGPAGAQKLVF

SEQ ID NO: 20 - protein, 15 aa, Artificial Sequence
  Synthetic
CASSPVPYSGNTIYF

SEQ ID NO: 21 - protein, 11 aa, Artificial Sequence
  Synthetic
CAVSKSDKIIF

SEQ ID NO: 22 - protein, 15 aa, Artificial Sequence
  Synthetic
CATSEGGSTGTEAFF

SEQ ID NO: 23 - protein, 13 aa, Artificial Sequence
  Synthetic
CALNRNTGNQFYF

SEQ ID NO: 24 - protein, 13 aa, Artificial Sequence
  Synthetic
CASSIPQGNGYTF

SEQ ID NO: 25 - protein, 275 aa, Artificial Sequence
  Synthetic
MLLLLVPVLEVIFTLGGTRAQSVTQLGSHVSVSEGALVLLRCNYSSSVPPYLFWYVQYPNQGLQLLLKYTSAATLVKGINGFEAEFKKSETSFHLTKPSAHMSDAAEYFCAVSVGPGNTGKLIFGQGTTLQVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 26 - protein, 308 aa, Artificial Sequence
  Synthetic
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSLGVLGLRYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 27 - protein, 274 aa, Artificial Sequence
  Synthetic
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAENSGGSNYKLTFGKGTLLTVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 28 - protein, 308 aa, Artificial Sequence
  Synthetic
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSWERAGKAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 29 - protein, 274 aa, Artificial Sequence
  Synthetic
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSVFIYSTFIFGSGTRLSVKPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 30 - protein, 307 aa, Artificial Sequence
  Synthetic
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSGRQETQYFGPGTRLLVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 31 - protein, 271 aa, Artificial Sequence
  Synthetic
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDAYTRQLTFGSGTQLTVLPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 32 - protein, 309 aa, Artificial Sequence
  Synthetic
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASGGPGANRPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 33 - protein, 277 aa, Artificial Sequence
  Synthetic
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSLWGSGYALNFGKGTSLLVTPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 34 - protein, 306 aa, Artificial Sequence
  Synthetic
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVEKGAQETQYFGPGTRLLVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 35 - protein, 271 aa, Artificial Sequence
  Synthetic
MTSIRAVFIFLWLQLDLVNGENVEQHPSTLSVQEGDSAVIKCTYSDSASNYFPWYKQELGKGPQLIIDIRSNVGEKKDQRIAVTLNKTAKHFSLHITETQPEDSAVYFCAALFGNEKLTFGTGTRLTIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 36 - protein, 310 aa, Artificial Sequence
  Synthetic
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCATLQGWSYNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 37 - protein, 268 aa, Artificial Sequence
  Synthetic
MKPTLISVLVIIFILRGTRAQRVTQPEKLLSVFKGAPVELKCNYSYSGSPELFWYVQYSRQRLQLLLRHISRESIKGFTADLNKGETSFHLKKPFAQEEDSAMYYCALSTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 38 - protein, 312 aa, Artificial Sequence
  Synthetic
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVLGPGTGGRGANYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 39 - protein, 271 aa, Artificial Sequence
  Synthetic
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATTIDGQKLLFARGTMLKVDLNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 40 - protein, 307 aa, Artificial Sequence
  Synthetic
MLLLLLLLGPAGSGLGAVVSQHPSRVICKSGTSVKIECRSLDFQATTMFWYRQFPKKSLMLMATSNEGSKATYEQGVEKDKFLINHASLTLSTLTVTSAHPEDSSFYICSASDRGSGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 41 - protein, 271 aa, Artificial Sequence
  Synthetic
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDAYNARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 42 - protein, 307 aa, Artificial Sequence
  Synthetic
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVVASGSVDTQYFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 43 - protein, 269 aa, Artificial Sequence
  Synthetic
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGPAGAQKLVFGQGTRLTINPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 44 - protein, 310 aa, Artificial Sequence
  Synthetic
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSPVPYSGNTIYFGEGSWLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 45 - protein, 271 aa, Artificial Sequence
  Synthetic
MLLLLVPVLEVIFTLGGTRAQSVTQLGSHVSVSEGALVLLRCNYSSSVPPYLFWYVQYPNQGLQLLLKYTSAATLVKGINGFEAEFKKSETSFHLTKPSAHMSDAAEYFCAVSKSDKIIFGKGTRLHILPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 46 - protein, 310 aa, Artificial Sequence
  Synthetic
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSEGGSTGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 47 - protein, 272 aa, Artificial Sequence
  Synthetic
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALNRNTGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 48 - protein, 308 aa, Artificial Sequence
  Synthetic
MSNQVLCCVVLCLLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIPQGNGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 49 - DNA, 828 bp, Artificial Sequence
  Synthetic
atgctcctgctgctcgtcccagtgctcgaggtgatttttaccctgggaggaaccagagcccagtcggtgacccagcttggcagccacgtctctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgctgtgagtgtaggtcctggcaacacaggcaaactaatctttgggcaagggacaactttacaagtaaaaccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 50 - DNA, 927 bp, Artificial Sequence
  Synthetic
atgggaatcaggctcctgtgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagtttgggggttctaggcctgcggtacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 51 - DNA, 825 bp, Artificial Sequence
  Synthetic
atggcaggcattcgagctttatttatgtacttgtggctgcagctggactgggtgagcagaggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagaatagtggaggtagcaactataaactgacatttggaaaaggaactctcttaaccgtgaatccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 52 - DNA, 927 bp, Artificial Sequence
  Synthetic
atgggccctgggctcctctgctgggtgctgctttgtctcctgggagcaggctcagtggagactggagtcacccaaagtcccacacacctgatcaaaacgagaggacagcaagtgactctgagatgctcttctcagtctgggcacaacactgtgtcctggtaccaacaggccctgggtcaggggccccagtttatctttcagtattatagggaggaagagaatggcagaggaaacttccctcctagattctcaggtctccagttccctaattatagctctgagctgaatgtgaacgccttggagctggacgactcggccctgtatctctgtgccagcagctgggaaagggcggggaaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 53 - DNA, 825 bp, Artificial Sequence
  Synthetic
atgatgaaatccttgagagttttactggtgatcctgtggcttcagttaagctgggtttggagccaacagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatgagcgtctttatttatagcacattcatctttgggagtgggacaagattatcagtaaaacctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 54 - DNA, 924 bp, Artificial Sequence
  Synthetic
atgggaatcaggctcctgtgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagctctgggaggcaagagacccagtacttcgggccaggcacgcggctcctggtgctcgaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 55 - DNA, 816 bp, Artificial Sequence
  Synthetic
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggacgcgtacacaaggcaactgacctttggatctgggacacaattgactgttttacctgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 56 - DNA, 930 bp, Artificial Sequence
  Synthetic
atgagcaaccaggtgctctgctgtgtggtcctttgtctcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtgggggaccgggggctaacaggccccagcattttggtgatgggactcgactctccatcctagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 57 - DNA, 834 bp, Artificial Sequence
  Synthetic
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggagcctttgggggtccgggtatgcactcaacttcggcaaaggcacctcgctgttggtcacaccccatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 58 - DNA, 921 bp, Artificial Sequence
  Synthetic
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgaaaagggcgcgcaagagacccagtacttcgggccaggcacgcggctcctggtgctcgaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 59 - DNA, 816 bp, Artificial Sequence
  Synthetic
atgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcactctttggaaatgagaaattaacctttgggactggaacaagactcaccatcatacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 60 - DNA, 933 bp, Artificial Sequence
  Synthetic
atgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtctccactctgacgatccagcgcacacagcaggaggactcggccgtgtatctctgtgccaccctacagggttggtcttataatgagcagttcttcgggccagggacacggctcaccgtgctagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 61 - DNA, 807 bp, Artificial Sequence
  Synthetic
atgaagcccaccctcatctcagtgcttgtgataatatttatactcagaggaacaagagcccagagagtgactcagcccgagaagctcctctctgtctttaaaggggccccagtggagctgaagtgcaactattcctattctgggagtcctgaactcttctggtatgtccagtactccagacaacgcctccagttactcttgagacacatctctagagagagcatcaaaggcttcactgctgaccttaacaaaggcgagacatctttccacctgaagaaaccatttgctcaagaggaagactcagccatgtattactgtgctctaagtactggaggcttcaaaactatctttggagcaggaacaagactatttgttaaagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 62 - DNA, 939 bp, Artificial Sequence
  Synthetic
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttttgggtcccgggacagggggacgaggagctaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 63 - DNA, 816 bp, Artificial Sequence
  Synthetic
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctaccactatagatggccagaagctgctctttgcaaggggaaccatgttaaaggtggatcttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 64 - DNA, 924 bp, Artificial Sequence
  Synthetic
atgctgctgcttctgctgcttctggggccagcaggctccgggcttggtgctgtcgtctctcaacatccgagcagggttatctgtaagagtggaacctctgtgaagatcgagtgccgttccctggactttcaggccacaactatgttttggtatcgtcagttcccgaaaaagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgcttctgacagggggtccggggagctgttttttggagaaggctctaggctgaccgtactggaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 65 - DNA, 816 bp, Artificial Sequence
  Synthetic
atggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggacgcatacaatgccagactcatgtttggagatggaactcagctggtggtgaagcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 66 - DNA, 924 bp, Artificial Sequence
  Synthetic
atgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgtggcaagcgggagtgtagatacgcagtattttggcccaggcacccggctgacagtgctcgaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 67 - DNA, 810 bp, Artificial Sequence
  Synthetic
atggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcagcccagcctggtgatacaggcctctacctctgtgcaggtcctgcgggagcccagaagctggtatttggccaaggaaccaggctgactatcaacccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 68 - DNA, 933 bp, Artificial Sequence
  Synthetic
atgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagccccgtaccctactctggaaacaccatatattttggagagggaagttggctcactgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 69 - DNA, 816 bp, Artificial Sequence
  Synthetic
atgctcctgctgctcgtcccagtgctcgaggtgatttttaccctgggaggaaccagagcccagtcggtgacccagcttggcagccacgtctctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgctgtgagtaagagtgacaagatcatctttggaaaagggacacgacttcatattctccccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 70 - DNA, 933 bp, Artificial Sequence
  Synthetic
atggcctccctgctcttcttctgtggggccttttatctcctgggaacagggtccatggatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtgagggggggagtacgggcactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 71 - DNA, 819 bp, Artificial Sequence
  Synthetic
atgaactattctccaggcttagtatctctgatactcttactgcttggaagaacccgtggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgaataggaacaccggtaaccagttctattttgggacagggacaagtttgacggtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 72 - DNA, 927 bp, Artificial Sequence
  Synthetic
atgagcaaccaggtgctctgctgtgtggtcctttgtctcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagtatcccacagggcaatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 73 - DNA, 16 bp, Artificial Sequence
  Synthetic
gagtgtaggtcctggc

SEQ ID NO: 74 - DNA, 28 bp, Artificial Sequence
  Synthetic
agtttgggggttctaggcctgcggtact

SEQ ID NO: 75 - DNA, 45 bp, Artificial Sequence
  Synthetic
tgtgctgtgagtgtaggtcctggcaacacaggcaaactaatcttt

SEQ ID NO: 76 - DNA, 39 bp, Artificial Sequence
  Synthetic
tgtgccagcagtttgggggttctaggcctgcggtacttc

SEQ ID NO: 77 - DNA, 16 bp, Artificial Sequence
  Synthetic
  misc_feature (6)..(11): “agaata” may or may not be present
tgcagagaatagtgga

SEQ ID NO: 78 - DNA, 42 bp, Artificial Sequence
  Synthetic
tgtgcagagaatagtggaggtagcaactataaactgacattt

SEQ ID NO: 79 - DNA, 22 bp, Artificial Sequence
  Synthetic
ctgggaaagggcggggaaagct

SEQ ID NO: 80 - DNA, 39 bp, Artificial Sequence
  Synthetic
tgtgccagcagctgggaaagggcggggaaagctttcttt

SEQ ID NO: 81 - DNA, 15 bp, Artificial Sequence
  Synthetic
gagcgtctttattta

SEQ ID NO: 82 - DNA, 39 bp, Artificial Sequence
  Synthetic
tgtgcaatgagcgtctttatttatagcacattcatcttt

SEQ ID NO: 83 - DNA, 19 bp, Artificial Sequence
  Synthetic
ccagctctgggaggcaaga

SEQ ID NO: 84 - DNA, 36 bp, Artificial Sequence
  Synthetic
tgtgccagctctgggaggcaagagacccagtacttc

SEQ ID NO: 85 - DNA, 16 bp, Artificial Sequence
  Synthetic
ggacgcgtacacaagg

SEQ ID NO: 86 - DNA, 36 bp, Artificial Sequence
  Synthetic
tgtgctacggacgcgtacacaaggcaactgaccttt

SEQ ID NO: 87 - DNA, 30 bp, Artificial Sequence
  Synthetic
ccagtgggggaccgggggctaacaggcccc

SEQ ID NO: 88 - DNA, 42 bp, Artificial Sequence
  Synthetic
tgtgccagtgggggaccgggggctaacaggccccagcatttt

SEQ ID NO: 89 - DNA, 19 bp, Artificial Sequence
  Synthetic
ggagcctttgggggtccgg

SEQ ID NO: 90 - DNA, 45 bp, Artificial Sequence
  Synthetic
tgtgcttataggagcctttgggggtccgggtatgcactcaacttc

SEQ ID NO: 91 - DNA, 19 bp, Artificial Sequence
  Synthetic
ttgaaaagggcgcgcaaga

SEQ ID NO: 92 - DNA, 39 bp, Artificial Sequence
  Synthetic
tgcagcgttgaaaagggcgcgcaagagacccagtacttc

SEQ ID NO: 93 - DNA, 12 bp, Artificial Sequence
  Synthetic
cagcactctttg

SEQ ID NO: 94 - DNA, 36 bp, Artificial Sequence
  Synthetic
tgtgcagcactctttggaaatgagaaattaaccttt

SEQ ID NO: 95 - DNA, 30 bp, Artificial Sequence
  Synthetic
tgccaccctacagggttggtcttataatga

SEQ ID NO: 96 - DNA, 42 bp, Artificial Sequence
  Synthetic
tgtgccaccctacagggttggtcttataatgagcagttcttc

SEQ ID NO: 97 - DNA, 11 bp, Artificial Sequence
  Synthetic
  misc_feature (6)..(6): “t” may or may not be present
ctaagtactgg

SEQ ID NO: 98 - DNA, 36 bp, Artificial Sequence
  Synthetic
tgtgctctaagtactggaggcttcaaaactatcttt

SEQ ID NO: 99 - DNA, 38 bp, Artificial Sequence
  Synthetic
gcgttttgggtcccgggacagggggacgaggagctaac

SEQ ID NO: 100 - DNA, 57 bp, Artificial Sequence
  Synthetic
tgcagcgttttgggtcccgggacagggggacgaggagctaactatggctacaccttc

SEQ ID NO: 101 - DNA, 16 bp, Artificial Sequence
  Synthetic
gctaccactatagatg

SEQ ID NO: 102 - DNA, 36 bp, Artificial Sequence
  Synthetic
tgtgctaccactatagatggccagaagctgctcttt

SEQ ID NO: 103 - DNA, 23 bp, Artificial Sequence
  Synthetic
gtgcttctgacagggggtccggg

SEQ ID NO: 104 - DNA, 39 bp, Artificial Sequence
  Synthetic
tgcagtgcttctgacagggggtccggggagctgtttttt

SEQ ID NO: 105 - DNA, 13 bp, Artificial Sequence
  Synthetic
ggacgcatacaat

SEQ ID NO: 106 - DNA, 36 bp, Artificial Sequence
  Synthetic
tgtgctacggacgcatacaatgccagactcatgttt

SEQ ID NO: 107 - DNA, 26 bp, Artificial Sequence
  Synthetic
cgttgtggcaagcgggagtgtagata

SEQ ID NO: 108 - DNA, 42 bp, Artificial Sequence
  Synthetic
tgcagcgttgtggcaagcgggagtgtagatacgcagtatttt

SEQ ID NO: 109 - DNA, 16 bp, Artificial Sequence
  Synthetic
gcaggtcctgcgggag

SEQ ID NO: 110 - DNA, 36 bp, Artificial Sequence
  Synthetic
tgtgcaggtcctgcgggagcccagaagctggtattt

SEQ ID NO: 111 - DNA, 22 bp, Artificial Sequence
  Synthetic
agcagccccgtaccctactctg

SEQ ID NO: 112 - DNA, 45 bp, Artificial Sequence
  Synthetic
tgtgccagcagccccgtaccctactctggaaacaccatatatttt

SEQ ID NO: 113 - DNA, 15 bp, Artificial Sequence
  Synthetic
tgagtaagagtgaca

SEQ ID NO: 114 - DNA, 33 bp, Artificial Sequence
  Synthetic
tgtgctgtgagtaagagtgacaagatcatcttt

SEQ ID NO: 115 - DNA, 25 bp, Artificial Sequence
  Synthetic
agtgagggggggagtacgggcactg

SEQ ID NO: 116 - DNA, 45 bp, Artificial Sequence
  Synthetic
tgtgccaccagtgagggggggagtacgggcactgaagctttcttt

SEQ ID NO: 117 - DNA, 14 bp, Artificial Sequence
  Synthetic
tctgaataggaaca

SEQ ID NO: 118 - DNA, 39 bp, Artificial Sequence
  Synthetic
tgtgctctgaataggaacaccggtaaccagttctatttt

SEQ ID NO: 119 - DNA, 21 bp, Artificial Sequence
  Synthetic
agtatcccacagggcaatggc

SEQ ID NO: 120 - DNA, 39 bp, Artificial Sequence
  Synthetic
tgtgccagtagtatcccacagggcaatggctacaccttc